SEQUENCE LISTING (26 sequences)

SEQ ID NO: 1 - DNA, 1362 bp, Infectious bursal disease virus
  source 1..1362: mol_type = genomic DNA
atgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagttacaactactgcaggctagtgagtcggagtctcacagtgaggtcaagcacactccctggtggcgtttatgcactaaacggcaccgtaaacgccgtgaccttccaaggaagcctgagtgaactgacagatgttagctacaatgggttgatgtctgcaacggccaacatcaacgacaaaattgggaatgtcctagtaggggaaggggtcaccgtcctcagcttacccacatcatatgatcttgggtatgtgaggcttggtgaccccattcctgctatagggcttgacccaaaaatggtagccacatgtgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgttccaaacaagcgtccaaggccttgtactgggcgctaccatctaccttataggctttgatgggactacagtaatcaccagagctgtggcctcagacaatgggctgactgccggcaccgacaatcttatgccattcaatcttgtgattccgaccaacgagataacccagccaatcacatccatcaaactggagatagtgacctccaaaagtggcggtcaggcaggggaccagatgtcatggtcggcaagtgggagcctagcagtgacaatccatggtggcaactatccaggggccctccgtcccgtcacactagtagcctacgaaagagtggcaacaggatccgtcgttacggtagccggggtgagcaacttcgagctgatcccaaatcctgaactagcaaagaacctggttacagaatacggccgatttgacccaggagccatgaactacacaaaattgatactgagtgagagggaccgtcttggcatcaagaccgtctggccaacaagggagtacactgactttcgtgagtacttcatggaggtggccgacctcaactctcccctgaagattgcaggagcatttggcttcaaagacataatccgggccataaggaggtga

SEQ ID NO: 2 - DNA, 1506 bp, synthetic construct
  source 1..1506: mol_type = other DNA
tgcagctcagtgcatgcacgctcattgcccatcgctatccctgcctctcctgctggcgctccccgggaggtgacttcaaggggaccgcaggaccacctcgggggtggggggagggctgcacacgcggaccccgctccccctccccaacaaagcactgtggaatcaaaaaggggggaggggggatggaggggcgcgtcacacccccgccccacaccctcacctcgaggtgagccccacgttctgcttcactctccccatctcccccccctccccacccccaattttgtatttatttattttttaattattttgtgcagcgatgggggcggggggggggggggcgcgcgccaggcggggcggggcggggccaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgaagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggggtttatatcttcccttctctgttcctccgcagccccc

SEQ ID NO: 3 - DNA, 302 bp, synthetic construct
  source 1..302: mol_type = other DNA
ctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 4 - DNA, 72 bp, synthetic construct
  source 1..72: mol_type = other DNA
ggagaggagggcggaaatcagtggaacacttctgccccctactggtatgcaacaggatcattagagaaatga

SEQ ID NO: 5 - DNA, 1410 bp, Infectious laryngotracheitis virus
  source 1..1410: mol_type = genomic DNA
atggctagcttgaaaatgctgatctgcgtgtgcgtggcaatcctgatcccatctaccctatctcaagattcacacggaattgccggaataatagaccctcgtgatacagccagcatggatgttggaaaaatctctttctccgaagccattgggtcgggggcaccgaaagaaccccagattagaaacagaatttttgcgtgctcatctccaactggcgccagtgttgcgaggcttgcccagccacgacattgtcaccgacatgccgattcgactaacatgactgaaggaattgccgtagtcttcaagcaaaacattgccccgtacgtctttaatgtgactctatactataaacatataaccacagttactacgtgggcattattctcaagaccccaaataacaaatgagtacgtgaccagggttccaatagactatcatgaaattgtcaggattgatcgatcgggagaatgctcatccaaagcaacgtatcataaaaatttcatgttttttgaagcttacgacaatgatgaacgagaaaaaaaattgcccctggttccatcactgttaagatcaactgtctccaaggcgtttcatacaactaactttactaagcgacatcaaaccctgggataccgaacgtctacatcggtcgactgtgttgtggaatatctacaggctagatctgtatacccgtatgattactttggaatggcgacaggtgatacagtagaaatttctcccttttataccaaaaacacgaccggaccaaggcgtcacagtgtctacagagactatagatttctcgaaatcgcaaattatcaagtcagggatttggaaaccggacaaataagaccccctaaaaaaagaaactttctaacagatgaacaattcactataggctgggatgcaatggaagaaaaggaatctgtatgtactctcagtaaatggattgaagtcccggaagcagttcgtgtttcgtacaaaaacagttaccacttttcacttaaagatatgactatgacgttctcgtccggaaaacaaccttttaacatcagcaggcttcatttggctgaatgcgttcctaccatagcttcggaggccatagatggcatctttgccagaaagtatagttcgactcatgtccgttctggggacatcgaatactatctcggtagtggcggatttctgatcgcatttcagaaactcatgagccatggcttggctgaaatgtacctagaagaggcacaaagacaaaatcatctcccgagagggagagagcgtcgccaaggcgacctctacaaatgtggtatggctgattatgatcatgaacagactgtgaggactgaggggcctgaaatgagccttgggactgtgaatcggccaataaggcctatttactcatcgcattag

SEQ ID NO: 6 - DNA, 541 bp, synthetic construct
  source 1..541: mol_type = other DNA
tactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcag

SEQ ID NO: 7 - DNA, 31 bp, synthetic construct
  source 1..31: mol_type = other DNA
cccccagcatacctaaataaataaaaaggcc

SEQ ID NO: 8 - DNA, 552 bp, synthetic construct
  source 1..552: mol_type = other DNA
agttattaatagtaatcaattacggggtcattagttcatagcccatatatggagttccgcgttacataacttacggtaaatggcccgccggctgaccgcccaacgacccccgcccattgacgtcaataatgacgtatgttcccatagtaacgccaatagggactttccattgacgtcaatgggtggagtatttacggtaaactgcccattggcagtacatcaagtgtatcatatgccaagtacgccccctattgacgtcaatgacggtaaatggatgcagtattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgt

SEQ ID NO: 9 - DNA, 30 bp, synthetic construct
  source 1..30: mol_type = other DNA
tgtcattctagagtcgacaagcttgggggc

SEQ ID NO: 10 - DNA, 99 bp, synthetic construct
  source 1..99: mol_type = other DNA
tcatttctctaatgatcctgttgcataccagtagggggcagaagtgttccactgatttccgccctcctctcctgcagccttattggccaaaacacacct

SEQ ID NO: 11 - DNA, 99 bp, synthetic construct
  source 1..99: mol_type = other DNA
ggagaggagggcggaaatcagtggaacacttctgccccctactggtatgcaacaggatcattagagaaatgagctcagtgcatgcacgctcattgccca

SEQ ID NO: 12 - DNA, 30 bp, synthetic construct
  source 1..30: mol_type = other DNA
gattacgaattcatggaagaaatttcctcc

SEQ ID NO: 13 - DNA, 24 bp, synthetic construct
  source 1..24: mol_type = other DNA
ggggaagtcttccggttaagggac

SEQ ID NO: 14 - DNA, 24 bp, synthetic construct
  source 1..24: mol_type = other DNA
ggtgcaattcgtaagaccgatggg

SEQ ID NO: 15 - DNA, 24 bp, synthetic construct
  source 1..24: mol_type = other DNA
agcaccatcgcattgtttcgtaga

SEQ ID NO: 16 - DNA, 24 bp, synthetic construct
  source 1..24: mol_type = other DNA
ctatctgtttccgtgccgctatcc

SEQ ID NO: 17 - DNA, 1623 bp, Newcastle disease virus
  source 1..1623: mol_type = genomic DNA
atgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatctttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtga

SEQ ID NO: 18 - DNA, 552 bp, synthetic construct; identical to SEQ ID NO: 8
  source 1..552: mol_type = other DNA

SEQ ID NO: 19 - DNA, 40 bp, synthetic construct
  source 1..40: mol_type = other DNA
caggatcattagagaaatgaggccaataaggctgcagtac

SEQ ID NO: 20 - DNA, 20 bp, synthetic construct
  source 1..20: mol_type = other DNA
ggcataagattgtcggtgcc

SEQ ID NO: 21 - DNA, 20 bp, synthetic construct
  source 1..20: mol_type = other DNA
ggtggttcgaatctggccgc

SEQ ID NO: 22 - DNA, 39 bp, synthetic construct
  source 1..39: mol_type = other DNA
tgatttccgccctcctctccttaaataccgcgtttggag

SEQ ID NO: 23 - DNA, 72 bp, synthetic construct; identical to SEQ ID NO: 4
  source 1..72: mol_type = other DNA

SEQ ID NO: 24 - DNA, 72 bp, synthetic construct
  source 1..72: mol_type = other DNA
tcatttctctaatgatcctgttgcataccagtagggggcagaagtgttccactgatttccgccctcctctcc

SEQ ID NO: 25 - DNA, 24 bp, synthetic construct
  source 1..24: mol_type = other DNA
ggggaagtcttccggttaagggac

SEQ ID NO: 26 - DNA, 24 bp, synthetic construct
  source 1..24: mol_type = other DNA
ggtgcaattcgtaagaccgatggg

(19) United States
(12) Patent Application Publication
(10) Pub. No.: US 2026/0183386 A1
(43) Pub. Date: Jul. 2, 2026

(54) RECOMBINANT MAREK'S DISEASE VIRUS AND USES THEREOF

(71) Applicant: CEVA SANTE ANIMALE, LIBOURNE (FR)

(72) Inventors: Kenta Sasaki, LIBOURNE (FR); Jina Lee, LIBOURNE (FR); Yasutoshi Komiya, LIBOURNE (FR); Yuichi Kawamura, LIBOURNE (FR)

(21) Appl. No.: 19/130,826

(22) PCT Filed: Nov. 17, 2023
(86) PCT No.: PCT/EP2023/082227

§ 371 Date: May 16, 2025

(30) Foreign Application Priority Data
Nov. 18, 2022 (EP) 22306708.3

(51) Int. Cl. A61K 39/245; A61K 39/00; A61P 31/22; C07K 14/005; C12N 7/00

(52) U.S. Cl. CPC A61K 39/245; A61P 31/22; C07K 14/005; C12N 7/00; A61K 2039/5256; A61K 2039/552; A61K 2039/70; C12N 2710/16022; C12N 2710/16034; C12N 2710/16321; C12N 2710/16343; C12N 2720/10022; C12N 2720/10034; C12N 2830/40

(57) ABSTRACT

The present invention relates to recombinant Marek's disease viruses containing, inserted into at least one insertion site, at least one recombinant nucleotide sequence encoding an antigen operably linked to a promoter and an insulator, their manufacture, compositions comprising the same, and the uses thereof.

### FIELD OF THE INVENTION

The present invention relates to novel recombinant Marek's disease virus (rMDV) capable of improving the expression of foreign antigen(s), and uses thereof. More particularly, the invention relates to novel rMDV comprising at least one foreign gene, whose expression is modulated by an insulator element. The invention also relates to uses of such rMDV for inducing protective immunity against avian pathogen(s) or disease(s).

### BACKGROUND OF THE INVENTION

Poultry meat and eggs are important food sources, whose consumption increases continually due to the growth of the human population and their great quality-price ratio. In order to ensure poultry health as well as food safety and security, poultry vaccine technology has become a worldwide concern.

Viral vectors expressing pathogen proteins are commonly used as poultry vaccines against targeted pathogens. Vaccines including such viral vectors induce expression of foreign pathogen proteins within infected hosts, which may lead to protective immunity.

Many different classes of viruses have been investigated as candidate vectors for vaccination of avian, such as adenoviruses, AAVs, fowlpox viruses, and avian herpes viruses. In particular, Marek disease virus (MDV) of serotypes 1, 2 and 3 (also known as herpesvirus of turkeys (HVT)) have been used as recombinant vectors to express antigens from various avian pathogens.

The most common problems encountered with recombinant viruses are the stability of the foreign antigen(s) within the vector and the level of expression of said antigen(s) to allow protective immunity against the corresponding pathogen(s). Therefore, there is still a need for vectors, in particular MDV vectors, able to efficiently and stably express foreign gene(s) and thereby to protect avian against pathogens.

### SUMMARY OF THE INVENTION

By working on improvement of vectors suitable for avian vaccination, the inventors have developed new rMDV for the insertion and expression of one or more foreign genes for use as efficient immunization vehicles to protect against a variety of avian pathogens. More particularly, the inventors have developed a recombinant Marek's disease virus wherein at least one foreign gene is associated with a particular insulator suitable to positively influence gene expression of the associated foreign gene, i.e., to improve stability and/or level of expression. The inventors have more particularly developed an insulator derived from beta-globin 3′hypersensitivity site 1 (3′HS1) and comprising a CTCF motif. An expression cassette including said insulator associated to a recombinant nucleotide sequence encoding an antigen may be stably introduced in an insertion site of the rMDV, allowing the production of significant amounts of corresponding foreign antigen(s) and the induction of protective immunity against corresponding avian pathogen(s). The rMDV of the present invention is particularly suited to be used in vaccine composition to immunize avian, such as poultry, against one or more avian pathogens.

It is therefore an object of the present invention to provide recombinant Marek's disease virus (rMDV) comprising, inserted into an insertion site, a recombinant nucleotide sequence encoding an antigen operably linked to a promoter and an insulator, wherein the insulator is positioned upstream of the promoter.

Monovalent and multivalent rMDVs may be developed, wherein at least one foreign gene is associated to an insulator.

In particular, the present invention provides a rMDV comprising a first recombinant nucleotide sequence encoding a first antigen inserted in a first insertion site, and a second recombinant nucleotide sequence encoding a second antigen inserted into a second insertion site, different from the first insertion site, wherein the first recombinant nucleotide sequence encoding an antigen is operably linked to a promoter and to an insulator positioned upstream of the promoter, and wherein the second recombinant nucleotide sequence is operably linked to a promoter. In such construction, the second recombinant nucleotide sequence is not associated to an insulator.

It is therefore an object of the present invention to provide multivalent rMDVs, wherein a single foreign gene is associated to an insulator which is positioned upstream of a promoter driving the expression of said foreign gene.

The invention also relates to a nucleic acid molecule comprising, consisting essentially of, or consisting of the genome of a recombinant MDV as defined above, and to a vector (such as a plasmid) containing such a nucleic acid.

The invention further relates to a host cell comprising a recombinant MDV or a nucleic acid or vector as defined above.

The invention also relates to a method for producing or replicating a recombinant MDV as defined above, comprising infecting a competent host cell with the recombinant MDV or a nucleic acid molecule as defined above, and collecting the rMDV.

A further object of the invention is a composition, such as a vaccine composition, comprising a recombinant MDV, a nucleic acid and/or a host cell as defined above, and optionally a suitable excipient and/or adjuvant.

A further object of the invention resides in a recombinant MDV, nucleic acid, host cell, composition or vaccine, as defined above, for use for vaccinating an avian, such as poultry, against at least one avian pathogen, and/or an associated disease(s).

A further object of the invention resides in a recombinant MDV, nucleic acid, host cell, composition or vaccine, as defined above, for use for inducing an early onset of immunity in an avian, such as a poultry, against at least one avian pathogen.

A further object of the invention resides in a recombinant MDV, nucleic acid, host cell, composition or vaccine, as defined above, for use for inducing protective immunity in an avian, such as a poultry, against at least one avian pathogen.

A further object of the invention resides in a method for vaccinating an avian comprising administering to the avian the composition, vaccine or recombinant MDV as defined above.

The invention also provides a vaccination kit for immunizing an avian, such as a poultry, against an avian pathogen, which comprises the following components:
a. an effective amount of a vaccine as defined above, and
b. a means for administering said vaccine to said avian.

The invention may be used with any avian, in particular poultry, such as chicken.

### LEGEND TO THE FIGURES

FIG. 1(a) illustrates the genome structures of recombinant HVT/IBDs, according to the prior art (FW169; FW260) and to embodiments of the present invention (FW285; FW311).

FIG. 1(b) shows a diagram of recombinant HVT/IBD (FW285) genome, showing the portion amplified in PCR reaction to confirm the genome structure of the virus.

FIG. 1(c) shows results of PCR analysis for FW285, confirming expression of IBDV VP2 protein. FW285 was harvested after virus purification and sampled for PCR analysis. M: BioMarker™ 10 Kb (BioVentures, Inc., #M10 KB), N.C.: FC126 (negative control) and P.C.: homology plasmid (positive control).

FIG. 2(a) is western blotting analysis showing the expression of VP2 protein in CEF cells infected with FW285, FW181 or FW169. After 3 days from the infection of each recombinant HVT into CEFs at MOI=0.1, each sample was harvested and underwent SDS-PAGE followed by western blot assay. To detect VP2 protein, anti-VP2 mouse mAb R63 was used as a 1st antibody at western blot. As shown in FIG. 3(a), a protein band of 40 kilodalton (kDa) was observed in the lane with FW285 and FW169, which was the expected size of the VP2 protein.

FIG. 2(b) visualizes the relative densities of bands from VP2 protein at western blot assay of FIG. 3(a) which were measured with respect to FW169, and are shown as bar graph. This quantification was conducted by ImageJ. Results demonstrates that FW285 shows a better expression of VP2 protein as compared to FW169.

FIG. 3 illustrates the average of anti-IBDV VP2 antibody titers in blood samples of SPF chickens vaccinated with recombinant HVT/IBD (FW169 or FW285), using a commercial IBD ELISA kit. NIC: non-infected control. Results show an earlier onset of immunity for FW285 (at 2 weeks) as compared to FW169 (at 3 weeks) and also a stronger immunity.

FIG. 4(a) shows a diagram of recombinant HVT/IBD-LT (FW311) genome, indicating the regions amplified in PCR reactions to confirm the genome structure of the virus.

FIG. 4(b) shows the result of PCR analysis for FW311. FW311 was harvested after the virus purification and sampled for the PCR analysis. N.C.: negative control and P.C. positive control.

FIG. 4(c) is western blotting analysis showing the expression of VP2 protein in CEF cells infected with FW260, FW311, FW181 or FW169. After 3 days from the infection of each recombinant HVT into CEFs at MOI=0.1, each sample was harvested and underwent SDS-PAGE followed by western blot assay. To detect VP2 protein, anti-VP2 mouse mAb R63 was used as a 1st antibody at western blot. As shown in FIG. 3(a), a protein band of 40 kilodalton (kDa) was observed for FW260, FW311 and FW169, which was the expected size of the VP2 protein

FIG. 5 visualizes the relative densities of bands from VP2 protein at western blot assay of FIG. 4(c), which were measured with respect to the vaccine control, FW169, and are shown as bar graph. This quantification was conducted by ImageJ. Results demonstrate that FW311 shows a better expression of VP2 protein as compared both to FW169 and FW260.

FIG. 6 illustrates the average of anti-IBDV VP2 antibody titers in blood samples of SPF chickens vaccinated with bivalent recombinant HVT/IBD-LT according to an embodiment of the present invention (FW311) as compared to FW169 and FW260, using a commercial IBD ELISA kit. NIC: non-infected control.

FIG. 7 illustrates the genome structures of a recombinant bivalent HVT/IBD-LT, according to a further embodiment of the present invention (FW313), as compared to genome structure of recombinant HVT/LTs a negative and positive controls (FW181; FW261).

FIG. 8(a) is western blotting analysis showing the expression of VP2 protein in CEF cells infected with FW313, FW261 or FW181. After 3 days from the infection of each recombinant HVT into CEFs at MOI=0.1, each sample was harvested and underwent SDS-PAGE followed by western blot assay. To detect VP2 protein, anti-VP2 mouse mAb R63 was used as a 1st antibody at western blot. A protein band of 40 kilodalton (kDa) was observed in the lane with FW313 and FW261, which was the expected size of the VP2 protein.

FIG. 8(b) visualizes the relative densities of bands from VP2 protein at western blot assay of FIG. 8(a), and are shown as bar graph. This quantification was conducted by ImageJ. Results demonstrate that FW313 shows a better expression of VP2 protein as compared to FW261.

FIG. 9 illustrates the genome structures of a recombinant HVT/NDV, according to a further embodiment of the present invention (FW348), as compared to genome structure of a recombinant HVT/NDV deprived of insulator, according to FW26.

FIG. 10 shows the NDV-F protein expression evaluated by Black plaque, with first antibody being anti-NDV-F mouse mAb (#77-2) and second antibody being biotinylated anti-mouse IgG.

FIG. 11 is western blotting analysis showing the expression of NDV-F protein in CEF cells infected with FW348, FW026 or FW169. After 3 days from the infection of each recombinant HVT into CEFs at MOI=0.01, each sample was harvested and underwent SDS-PAGE followed by western blot assay. To detect NDV-F protein, anti-NDV-F mouse mAb (#77-2) was used as a 1st antibody at western blot. A protein band of 60 kilodalton (kDa) was observed in the lane with FW348 and FW026, which was the expected size of the NDV-F protein.

### DETAILED DESCRIPTION OF THE INVENTION

The present invention generally relates to recombinant Marek's disease viruses containing an expression cassette comprising a recombinant nucleotide sequence operably linked to a promoter and an insulator sequence, their manufacture, composition comprising the same, and the uses thereof, in particular to immunize avian against avian pathogen(s). The rMDV of the invention are particularly stable, show good expression of the foreign gene(s) in vitro and provide effective immune-protection to avian. Therefore, rMDV of the invention are particularly suited for the generation of effective vaccines. Indeed, the rMDV of the invention show strong expression of the foreign gene(s), allowing an efficient onset of immunity (OOI), while preserving the stability and efficacy of the vaccines over time.

### Definitions

The present invention will be best understood by reference to the following definitions:

The term “recombinant” in relation to a sequence, designates a sequence, nucleic acid or unit which does not exist naturally and/or which has been engineered using recombinant DNA technology (also called gene cloning or molecular cloning).

The term “recombinant”, in relation to a virus, refers to the genome of a virus which has been modified by insertion of at least one nucleotide sequence (e.g., DNA, such as a gene) which is not found naturally in the genome of said virus, or which is found naturally in said genome but in a different form or at a different position. It will be understood that the recombinant virus can be manufactured by a variety of methods such as recombinant DNA technology as described therein and, once made, can be reproduced without further use of recombinant DNA technology.

In the present description, the terms “nucleic acid”, “nucleic sequence,” and “nucleotide sequence” refer to a nucleic acid molecule having a determined sequence, which may be deoxyribonucleotides and/or ribonucleotides. The nucleotide sequence may be first prepared by e.g., recombinant, enzymatic and/or chemical techniques, and subsequently replicated in a host cell or an in vitro system. A nucleotide sequence preferentially comprises an open reading frame encoding a molecule (e.g. a peptide or protein). The nucleotide sequence may contain additional sequences such as a promoter, a transcription terminator, a signal peptide, an IRES, etc.

In the present description, the terms “polypeptide”, “peptide,” and “protein” refer to any molecule comprising a polymer of at least 10 consecutive amino acids.

An “immunogenic fragment” or “antigenic fragment” of an antigen, peptide or protein designates any fragment which can elicit an immune response, preferably any fragment which contains an epitope, preferably an antigen-specific epitope. Immunogenic fragments generally contain from 5 to 50 consecutive amino acid residues of an antigen, such as from 5 to 40, or from 10 to 40, or 10-30, 10-25, or 10-20.

The term “avian” or “avian species” is intended to encompass all kinds of avian such as birds of the class of Aves, i.e., vertebrate animals which are feathered, winged, bipedal, endothermic and egg-laying. In the context of the invention, avian or avian species refer more particularly to birds with economical and/or agronomical interests, such as poultry, (such as chickens and turkeys), waterfowl poultry (such as ducks and geese) and ornamental birds (such as swans and psittacines).

The term “vaccine” or “vaccine composition” as used herein designates an agent which may be used to cause, stimulate or amplify an immune response in an organism.

The term “multivalent”, as used herein in relation to a recombinant virus or vaccine refers to a recombinant virus or vaccine which comprises at least two recombinant nucleotide sequences or antigens, said sequences or antigens being the same or different, and from a same or a different pathogen.

### Recombinant MDV

The Marek's disease virus of the invention includes, but is not limited to, serotype 1 Marek's disease virus, preferably the CV1988/Rispens strain, serotype 2 Marek's disease virus, preferably the SB1 strain, and serotype 3 Marek's disease virus, preferably the herpes virus of turkey (HVT). Preferred Marek's disease virus of the invention are derived from serotypes or strains that are non-pathogenic to target avian species.

It is the purpose of the present invention to provide rMDV, which comprises inserted into an insertion site, a recombinant nucleotide sequence encoding an antigen, said recombinant nucleotide sequence being operably linked to a promoter and an insulator, wherein the insulator is positioned upstream of the promoter.

The presence of the insulator positioned upstream of the promoter driving the expression of the recombinant nucleotide sequence allows to favor the expression of said recombinant nucleotide sequence leading to a strong expression of corresponding antigen.

The rMDV of the invention allows an increased production of the antigen as compared to the production of same antigen by a rMDV deprived of insulator.

In a particular embodiment, an early onset of immunity and/or a strong onset of immunity is observed in avian vaccinated with the rMDV of the invention. In particular, an earlier onset of immunity may be observed, as compared to onset of immunity in avian vaccinated with a same vector deprived of such an insulator.

In the context of the present invention, the “onset of immunity (OOI)” refers to the timepoint, usually described in days or weeks after vaccination, when an active immunity allowing protection of the vaccinated avian against an avian pathogen or disease is obtained. An “early onset of immunity” refers to a protection reached at least 2 days earlier than OOI obtained with a reference vaccine (e.g., same vector, same antigen(s), same insertion site(s), but deprived of insulator), preferably at least 4 days earlier, 6 days earlier, more preferably at least 1 week, earlier. For instance, an early OOI with a rMDV/ND according to the invention may correspond to an immunity acquired about 3 weeks after vaccination, while corresponding reference vaccine requires 4 weeks to induce a complete protection (Palya V, et al. Vet Immunol Immunopathol. 2014. PMID: 24368086).

According to the invention, the rMDV may comprise one or more recombinant nucleotide sequences.

In a particular embodiment, the rMDV comprises a first recombinant nucleotide sequence encoding a first antigen inserted in a first insertion site, and a second recombinant nucleotide sequence encoding a second antigen inserted into a second insertion site, different from the first insertion site, wherein the first recombinant nucleotide sequence encoding an antigen is operably linked to a promoter and to an insulator positioned upstream of the promoter, and wherein the second recombinant nucleotide sequence is operably linked to a promoter. That is to say that the second recombinant nucleotide sequence is not associated to an insulator.

Multivalent rMDV of the invention allow efficient and stable expression of both recombinant nucleotide sequences.

### Insulator

According to the invention, the rMDV comprises at least one insulator, associated to a recombinant nucleotide sequence of interest, said insulator being position upstream of the promoter driving the expression of said recombinant nucleotide sequence.

Indeed, by working on the development of improved rMDV, able to stably express recombinant antigen(s), the inventors have shown that an insulator may be advantageously associated to a recombinant nucleotide sequence in order to provide efficient expression of the corresponding antigen. The inventors have developed rMDV integrating such an insulator that expresses the associated recombinant nucleotide sequence in a stably manner, i.e., even after 10 passages in cell culture, preferably 15 or 20 passages.

As used herein, the term “insulator” or “insulator element” refers to a DNA sequence which insulates the transcription of gene(s) placed within its range of action, thereby protecting the transcription of said gene(s) from negative effects such as enhancer-blocking activity and chromatin position effects. An insulator is able to protect a gene of interest from inappropriate signals originating from the surrounding environment by acting as a physical barrier or boundary. The nucleotide sequence or gene “associated” to an insulator refers to the nucleotide sequence or gene placed within the range of action of said insulator.

In the context of the invention “positioned upstream” means positioned at or toward the 5′ end of a gene of interest in the coding strand, with respect to the transcription direction. When considering double-stranded DNA, “upstream” is usually toward the 5′ end of the coding strand for the gene of interest, and “downstream” is toward the 3′ end. Some genes of a same DNA molecule may be transcribed in opposite directions. This means the upstream and downstream areas of the DNA molecule may change depending on the gene of interest.

The insulator of the invention is a DNA sequence introduced in an expression cassette, upstream of the promoter driving the expression of the recombinant nucleotide sequence of interest, to prevent or decrease interference of the viral genome and/or of other recombinant expression cassette(s), on the expression of the recombinant nucleotide sequence of interest. The insulator may further contribute to protect recombinant nucleotide sequence(s) from integration side effects, which may be mediated by cis-acting elements present in viral genome and lead to deregulated expression of transferred sequences. In particular, the insulator allows to prevent viral sequences from potentially interrupting the promoter activity, without intervening with its activity.

In particular embodiment, the insulator of the present invention allows to stabilize and/or increase the expression of the associated antigen.

Preferably, the insulator is not obtained or derived from an avian insulator. More preferably, the insulator is obtained or derived from a mammalian insulator, preferably a non-human mammalian insulator, such as a murine insulator.

Advantageously, the insulator comprises one or more CCCTC-binding factor (CTCF) motif(s). In a particular embodiment, the insulator comprises a single CTCF motif.

According to the invention, the insulator is preferably derived from a 3′hypersensitivity site 1 (3′HS1) insulator of the beta-globin locus. More particularly, the insulator may be derived from the 3′HS1 insulator elements described in Farrell (Farell et al., Molecular and cellular biology, 2002, vol. 22 (11), p. 3820-3831).

Preferably, the insulator comprises or consists of a functional fragment of a murine 3′hypersensitivity site 1 (m3′HS1) insulator. In the context of the present invention, a “functional fragment of an insulator” refers to a fragment which retains an insulator activity. The skilled person knows how to confirm insulator activity of an insulator's fragment. For instance, two rHVT expressing VP2 protein are constructed, which have an insulator's fragment sequence at 5′-end (e.g., linked with Bac promoter driving the expression of VP2 protein) or no insulator's fragment sequence (i.e., negative control). The expression level of VP2 protein by these two rHVT is compared. The insulator activity of the insulator's fragment is confirmed if VP2 expression by rHVT comprising insulator's fragment is higher than VP2 expression by rHVT deprived of insulator's fragment. The functional fragment of m3′HS1 advantageously comprises, consists essentially of, or consists of the nucleotide sequence of SEQ ID NO: 4 or a nucleotide sequence having at least 90%, preferably at least 95, 96, 97, 98 or 99% identity to SEQ ID NO:4 and retaining an insulator activity. Advantageously, the functional fragment of m3′HS1 comprises at least 90%, preferably at least 95, 96, 97, 98 or 99% identity to SEQ ID NO:4, including a CTCF motif and retains an insulator activity. In a particular embodiment, the insulator comprises or consists of the nucleotide sequence as set forth in SEQ ID NO:4.

SEQ ID NO: 4:
GGAGAGGAGGGCGGAAATCAGTGGAACACTTCTGCCCCCTACTGGTA
TGCAACAGGATCATTAGAGAAATGA

The insulator as set forth in SEQ ID NO: 4 comprises 72 nucleotides (herein after “3′HS1-72 insulator”), wherein the CTCF motif is located between positions 30 and 45 of SEQ ID NO: 4.

The inventors have shown that the 3′HS1-72 insulator, derived from the mouse beta-globin locus, can be used with success to improve recombinant nucleotide sequence(s) expression in cells. The 3′HS1-72 insulator has been shown to block the actions of enhancer elements in addition to functioning as a physical boundary that can prevent the spread of gene silencing.

The 3′HS1-72 insulator is particularly suited to be used in rHVT. It is therefore an object of the present invention to provide a rHVT comprising inserted into an insertion site, a recombinant nucleotide sequence encoding an antigen operably linked to a promoter and an insulator, wherein the insulator is positioned upstream of the promoter and comprises or consists of a nucleotide sequence having at least 90%, preferably at least 95, 96, 97, 98 or 99% identity to the full length sequence as set forth in SEQ ID NO:4. and retaining an insulator activity.

### Regulatory Sequences

According to the invention, the rMDV vector, such as a rHVT vector, comprises one or more insulator as described herein. The insulator is positioned upstream of the promoter driving the expression of the at least one recombinant nucleotide sequence placed under the control of said promoter. The insulator element can directly link to the promoter sequence.

The promoter may be either a synthetic or natural, endogenous or heterologous promoter. Any promoter may in principle be used, as long as it can effectively function in the target cells or host. In this regard, the promoter may be eukaryotic, prokaryotic, viral or synthetic promoter, capable of directing gene transcription in avian cells in the context of a recombinant vector.

If the rMDV comprises two or more recombinant nucleotide sequences, each recombinant nucleotide sequence may be operably linked to a promoter, which may be the same or different from each other. In a particular embodiment, each recombinant nucleotide sequence is operably linked to a different promoter.

Preferentially, the promoter is selected from a Pec promoter, a Cytomegalovirus (CMV) immediate early 1 (ie1) promoter, particularly a Murine Cytomegalovirus (Mcmv) ie1 promoter or a Human Cytomegalovirus (Hcmv) promoter, a chicken beta-actin (Bac) promoter, a Simian virus 40 (SV40) promoter, and a Rous Sarcoma virus (RSV) promoter, or any fragments thereof which retain a promoter activity.

The recombinant nucleotide sequence may be further operably linked to regulatory sequences, such as a polyadenylation signal. The insulator can then directly linked to the polyadenylation signal. In this particular embodiment, the insulator is positioned downstream of the polyadenylation signal.

The polyadenylation signal may be either a synthetic or natural, endogenous or heterologous polyadenylation signal. Any polyadenylation signal may in principle be used, as long as it can effectively function in the target cells or host. In this regard, the polyadenylation signal may be eukaryotic, prokaryotic, viral or synthetic polyadenylation signal, capable of stabilizing mRNAs and enhancing transcription termination in avian cells.

If the rMDV comprises two or more recombinant nucleotide sequences, each recombinant nucleotide sequence may be operably linked to a polyadenylation signal, which may be the same or different from each other.

The polyadenylation signal sequence is a base sequence comprising AATAAA, ATTAAA or a modified sequence thereof. Preferentially, the polyadenylation signal is derived from bovine growth hormone, simian virus 40 late and early region, rabbit beta-globin, mouse or human immunoglobulins, polyoma virus late region.

### Recombinant Nucleotide Sequences Encoding an Antigen

The recombinant nucleotide sequence(s) may encode any polypeptide of interest, such as antigens, cytokines, hormones, or adjuvants, for instance.

In particular, the recombinant nucleotide sequence(s) may encode an antigen from an avian pathogen or an antigenic fragment thereof.

The recombinant nucleotide sequence(s) may be derived or obtained from any pathogenic organism capable of causing an infection in an avian species. Examples of pathogens that cause infection in avian include viruses, bacteria, fungi, and protozoa.

Antigens may be any immunogenic peptides or proteins of a pathogen, such as a peptide or protein selected from or derived from surface proteins, secreted proteins or structural proteins of said pathogen, or antigenic fragments thereof.

Preferred recombinant nucleotide sequences for use in the present invention encode an antigen from avian influenza virus, avian paramyxovirus type 1 (also called Newcastle disease virus (NDV)), avian metapneumovirus, Marek's disease virus, Gumboro disease virus (also called infectious bursal disease virus (IBDV)), Infectious laryngotracheitis virus (ILVT), Infectious bronchitis virus (IBV), Escherichia coli, Salmonella, Pasteurella multocida, Riemerella anatipestifer, Ornithobacterium rhinotracheale, Mycoplasma gallisepticum, Mycoplasma synoviae, Mycoplasmas microorganisms infecting avian species, and/or coccidian.

Preferentially, the antigen(s) is/are selected from a F protein of NDV, a VP2 protein of IBDV, a gB protein of ILTV, a 40K protein of Mycoplasma gallisepticum, and a surface protein hemagglutinin (HA) of the avian influenza virus, or immunogenic fragments thereof.

When two or more recombinant nucleotide sequences are inserted in the rMDV, various combinations of antigens may be considered. Preferably, the two or more recombinant nucleotide sequences encode different antigens, more preferably from different pathogens.

In an embodiment, the recombinant MDV of the invention contains a recombinant nucleotide sequence encoding a VP2 protein of IBDV or an immunogenic fragment thereof.

In another embodiment, the recombinant MDV of the invention contains a nucleotide sequence encoding a VP2 protein of IBDV or an immunogenic fragment thereof and a nucleotide sequence encoding a gB protein of ILTV or an immunogenic fragment thereof.

In an embodiment, the recombinant MDV of the invention contains a recombinant nucleotide sequence encoding a F protein of NDV or an immunogenic fragment thereof.

In another embodiment, the recombinant MDV expresses two or more antigens from a same pathogen. Said antigens may be the same or different.

In a further embodiment, a recombinant nucleotide sequences encodes an active molecule such as a cytokine or immunomodulator, an adjuvant, a hormone, an antiparasitic agent, an antibacterial agent, and the like and the other recombinant nucleotide sequences encodes an antigen as defined above.

According to a further embodiment, three or more recombinant nucleotide sequences may be inserted into the viral genome.

### Insertion Sites

According to the invention, the recombinant nucleotide sequence, the promoter and optionally the insulator are inserted into an insertion site of the MDV.

Preferably, the insertion site(s) is/are located in non-coding regions of the viral genome.

The term “non-coding region” is well known in the art and refers to any region of a viral genome which does not encode a protein.

Preferably, the insertion site(s) may be selected from non-coding regions between UL43 and UL47, between UL55 and SORF4 and between US1 and US3. Particularly, insertion site(s) may be selected from non-coding regions located between UL44 and UL45, between UL45 and UL46, between UL55 and SORF4, between US10 and SORF3, and between SORF3 and US2. In particular, the insertion site(s) is selected from non-coding regions located between UL44 and UL45, between UL45 and UL46, and between SORF3 and US2.

Recombinant MDV of the invention may be prepared from any MDV, preferably non-pathogenic MDV. In particular, the rMDV is a recombinant HVT. Example of suitable HVT is the FC126 strain. The genomic sequence of the FC126 strain is available in the art (Afonso et al., supra; Kingham et al. supra), which reports the nucleotide sequence of the FC126 reference strain, as well as the location of most ORFs within said genome.

By reference to a FC126 complete genome (GenBank: AF291866.1), the non-coding region between UL44 (HVT052) and UL45 (HVT053) corresponds preferably to nucleotides 94243-94683 of the HVT genome, the non-coding region between UL45 (HVT053) and UL46 (HVT054) corresponds preferably to nucleotides 95323-95443 of the HVT genome, the non-coding region between UL55 (HVT065) and LORF4 (HVT066) corresponds to nucleotides 112010-112207 of the HVT genome, the non-coding region between US10 (HVT086) and SORF3 (HVT087) corresponds to nucleotides 138688-138825 of the HVT genome, and the non-coding region between SORF3 (HVT087) and US2 (HVT088) corresponds to nucleotides 139867-140064 of the HVT genome.

### Monovalent Constructions

An object of the present invention relates to a rMDV comprising a single foreign antigen operably linked to a promoter and an insulator positioned upstream of the promoter. That is to say, that the present invention relates to a rMDV comprising a single recombinant nucleotide sequence encoding a single foreign antigen.

Advantageously, a single insulator is positioned upstream of the promoter. However, it is possible to use two, three or more insulators associated to a same antigen. Preferably, said plurality of insulator are grouped and positioned one after the other, all upstream of the promoter.

Advantageously, the recombinant nucleic acid sequences (i.e., recombinant antigen, promoter and insulator) are inserted in the insertion site located in non-coding between UL45 and UL46, or between UL44 and UL45, or between SORF3 and US2.

In a particular embodiment, the recombinant nucleic acid sequences are inserted in the insertion site located in the non-coding region between UL45 and UL46.

Advantageously, the foreign antigen encodes an antigenic peptide selected from the F protein of NDV, the VP2 protein of IBDV, the gB protein of ILTV, the 40K protein of Mycoplasma galisepticum, and the surface protein HA of the avian influenza virus, or antigenic fragments thereof.

Among the plurality of possible embodiments based on the preferred insertion sites and preferred recombinant nucleotide sequences, the inventors have surprisingly found that constructions comprising a recombinant nucleotide sequence encoding a VP2 protein of IBDV or an antigenic fragment thereof present a high level of stability and allows a great expression of said antigen. The inventors have further shown that such constructions may allow an early onset of immunity against corresponding pathogen.

It is therefore an object of the present invention to propose a monovalent rMDV, preferably a rHVT, comprising in an insertion site selected from the non-coding region between UL45 and UL46 and the non-coding region between UL44 and UL45, a recombinant nucleotide sequence encoding a VP2 protein of IBDV or an antigenic fragment thereof operably linked to a promoter, and a m3′HS1 insulator, or a functional fragment thereof, positioned upstream of the promoter.

In a particular embodiment, the rHVT comprises in an insertion site located in the non-coding region between UL45 and UL46, a recombinant nucleotide sequence encoding a VP2 protein of IBDV or an antigenic fragment thereof operably linked to a Bac promoter, and a functional fragment of m3′HS1 insulator positioned upstream of the Bac promoter, wherein the functional fragment of m3′HS1 insulator preferably comprises or consists of a nucleotide sequence having a CTCF motif and at least 90% preferably at least 95, 96, 97, 98 or 99% identity to the full length sequence as set forth in SEQ ID NO:4, and retaining an insulator activity.

In another embodiment, the rHVT comprises in an insertion site located between UL44 and UL45, a recombinant nucleotide sequence encoding a VP2 protein of IBDV or an antigenic fragment thereof operably linked to a Murine Cytomegalovirus (Mcmv) immediate-early (ie)1 promoter, and a functional fragment of m3′HS1 insulator positioned upstream of the Mcmv(ie)1 promoter, wherein the functional fragment of m3′HS1 insulator preferably comprises or consists of a nucleotide sequence having a CTCF motif and at least 90%, preferably at least 95, 96, 97, 98 or 99% identity to the full length sequence as set forth in SEQ ID NO:4, and retaining an insulator activity.

It is another object of the invention to propose a rMDV, in particular a rHVT, comprising in an insertion site located between SORF3 and US2, a recombinant nucleotide sequence encoding a VP2 protein of IBDV or an antigenic fragment thereof operably linked to a promoter, in particular a Mcmvie1 promoter, and a functional fragment of m3′HS1 insulator positioned upstream of the promoter, wherein the functional fragment of m3′HS1 insulator preferably comprises or consists of a nucleotide sequence having a CTCF motif and having at least 90%, preferably at least 95, 96, 97, 98 or 99% identity to the full length sequence as set forth in SEQ ID NO:4, and retaining an insulator activity.

It is a particular object of the present invention to provide a rHVT comprising a nucleotide sequence encoding the VP2 protein of IBDV, preferably of SEQ ID NO:1 or having at least 80, 85, 90, 95, 96, 97, 98 or 99% identity to the full length sequence as set forth in SEQ ID NO:1, located under the control of Bac promoter, preferably of SEQ ID NO:2 or having at least 80, 85, 90, 95, 96, 97, 98 or 99% identity to the full length sequence as set forth in SEQ ID NO:2, flanked with the insulator sequence of SEQ ID NO:4 or having at least 90, 95, 96, 97, 98 or 99% identity to the full length sequence as set forth in SEQ ID NO:4, at the 5′-end and SV40 poly adenylation signal, preferably of SEQ ID NO:3 or having at least 80, 85, 90, 95, 96, 97, 98 or 99% identity to the full length sequence as set forth in SEQ ID NO:3, at the 3′-end, inserted into the intergenic region between HVT053 (UL45) and HVT054 (UL46) (FW285).

It is another object of the present invention to propose a monovalent rMDV, preferably a rHVT, comprising in an insertion site selected from the non-coding region between UL45 and UL46 and the non-coding region between UL44 and UL45, a recombinant nucleotide sequence encoding a F protein of NDV or an antigenic fragment thereof operably linked to a promoter, and a m3′HS1 insulator, or a functional fragment thereof, positioned upstream of the promoter.

In a particular embodiment, the rHVT comprises in an insertion site located in the non-coding region between UL45 and UL46, a recombinant nucleotide sequence encoding a F protein of NDV or an antigenic fragment thereof operably linked to a Bac promoter, and a functional fragment of m3′HS1 insulator positioned upstream of the Bac promoter, wherein the functional fragment of m3′HS1 insulator preferably comprises or consists of a nucleotide sequence having a CTCF motif and at least 90% preferably at least 95, 96, 97, 98 or 99% identity to the full length sequence as set forth in SEQ ID NO:4, and retaining an insulator activity.

It is a particular object of the present invention to provide a rHVT comprising a nucleotide sequence encoding the F protein of NDV, preferably of SEQ ID NO:17 or having at least 80, 85, 90, 95, 96, 97, 98 or 99% identity to the full length sequence as set forth in SEQ ID NO:17, located under the control of Bac promoter, preferably of SEQ ID NO:2 or having at least 80, 85, 90, 95, 96, 97, 98 or 99% identity to the full length sequence as set forth in SEQ ID NO:2, flanked with the insulator sequence of SEQ ID NO:4 or having at least 90, 95, 96, 97, 98 or 99% identity to the full length sequence as set forth in SEQ ID NO:4, at the 5′-end and SV40 poly adenylation signal, preferably of SEQ ID NO:3 or having at least 80, 85, 90, 95, 96, 97, 98 or 99% identity to the full length sequence as set forth in SEQ ID NO:3, at the 3′-end, inserted into the intergenic region between HVT053 (UL45) and HVT054 (UL46) (FW348).

### Multivalent Constructions

A further object of the present invention relates to a multivalent rMDV, comprising two or more recombinant nucleotide sequences encoding antigens, wherein at least one recombinant nucleotide sequence is associated to an insulator as described above. Preferably, the insulator comprises or consists of a functional fragment of m3′HS1 insulator having at least 90%, 95%, 98%, 99% with the nucleotide sequence as set forth in SEQ ID NO:4, including its CTCF motif, and retaining an insulator activity, or having exactly the nucleotide sequence as set forth in SEQ ID NO:4.

Two or more recombinant nucleotide sequences may be associated to an insulator. In particular, each recombinant nucleotide sequence may be associated to an insulator. Preferably, only one of the plurality of recombinant nucleotide sequences is associated to an insulator.

Preferably, the two or more recombinant nucleotide sequences encode different antigens. More preferably, the two or more recombinant nucleotide sequences encode different antigens from different pathogens.

The two or more recombinant nucleotide sequences may be inserted in same insertion sites or in different insertion sites. Preferably, the two or more recombinant nucleotide sequences are inserted in at least two different insertion sites. More preferably, each recombinant nucleotide sequence is inserted in a different insertion site.

In particular, the combination of insertion sites is selected among the non-coding regions located between UL44 and UL45, between UL45 and UL46, between UL55 and SORF4, between US10 and SORF3, and between SORF3 and US2, preferably among the non-coding regions located between UL44 and UL45, between UL45 and UL46, and between SORF3 and US2.

In a particular embodiment, the multivalent rMDV comprising one or more recombinant nucleotide sequences encoding an antigen, comprises only one cassette expression comprising an insulator as described above. That is to say that only one recombinant nucleotide sequence encoding antigen is associated to an insulator.

It is therefore an object of the present invention to propose a rMDV, in particular a rHVT comprising a first recombinant nucleotide sequence encoding a first antigen inserted in a first insertion site, and a second recombinant nucleotide sequence encoding a second antigen inserted into a second insertion site, different from the first insertion site, wherein the first recombinant nucleotide sequence encoding an antigen is operably linked to a promoter and to an insulator positioned upstream of the promoter, and wherein the second recombinant nucleotide sequence is operably linked to a promoter. The second expression cassette, comprising the second recombinant nucleotide sequence is free (i.e. deprived) of insulator. Advantageously, the first recombinant nucleotide sequence encodes a first antigen and the second recombinant nucleotide sequence encodes a second antigen different from the first antigen.

The multivalent rMDV may comprise a first recombinant nucleotide sequence inserted in the non-coding region located between UL44 and UL45, and a second recombinant nucleotide sequence inserted in the non-coding region located between UL45 and UL46, or the reverse.

Alternatively, the multivalent rMDV may comprise a first recombinant nucleotide sequence inserted in the non-coding region located between UL44 and UL45, and a second recombinant nucleotide sequence inserted in the non-coding region located between SORF3 and US2, or the reverse.

Alternatively, the multivalent rMDV may comprise a first recombinant nucleotide sequence inserted in the non-coding region located between UL45 and UL46, and a second recombinant nucleotide sequence inserted in the non-coding region located between SORF3 and US2, or the reverse.

Preferentially, the two or more recombinant nucleotide sequences encoding antigens are under the control of different promoters.

Advantageously, one recombinant nucleotide sequence encodes a VP2 protein of IBDV or an antigenic fragment thereof, and another recombinant nucleotide sequence encodes a gB protein of ILTV or an antigenic fragment thereof.

In a particular embodiment, the recombinant nucleotide sequence associated to an insulator according to the invention, encodes a VP2 protein of IBDV or an antigenic fragment thereof.

Among the plurality of possible embodiments based on the combinations of insertion sites and recombinant nucleotide sequences to be associated with the insulator, and optionally the preferred promoters, the inventors have surprisingly found that particular combinations lead to rMDV, in particular rHVT, with a high level of stability and a great expression level of both antigens. Such rMDV, and particularly such rHVT, are particularly suited for preparing improved multivalent vaccines. In particular, the inventors have shown that the use of an insulator as described above in combination with recombinant nucleotide sequence encoding a VP2 protein of IBDV or an antigenic fragment thereof, in a multivalent rMDV allows to stably and efficiently express the VP2 antigen without impairing the expression of the second antigen.

It is therefore an object of the present invention to provide a multivalent rMDV, preferably a multivalent rHVT, comprising in a first insertion site a first recombinant nucleotide sequence encoding a VP2 protein of IBDV or an antigenic fragment thereof operably linked to a promoter, and a m3′HS1 insulator, or a functional fragment thereof, positioned upstream of the promoter and, in a second insertion site a second recombinant nucleotide sequence encoding a second antigen different from VP2 antigen, operably linked to a promoter, wherein the first and second insertions are different and selected from the non-regions located between UL45 and UL46, between UL44 and UL45, and between SORF3 and US2.

In a particular embodiment, the multivalent rHVT comprises in a first insertion site located in the non-coding region between UL45 and UL46, a first recombinant nucleotide sequence encoding a VP2 protein of IBDV or an antigenic fragment thereof operably linked to a promoter, preferably a Bac promoter, and a m3′HS1 insulator, or a functional fragment thereof, positioned upstream of the Bac promoter and, in a second insertion site located in the non-coding region between UL44 and UL45, a second recombinant nucleotide sequence encoding a gB protein of ILTV or an antigenic fragment thereof operably linked to a promoter, preferably a Mcmv ie1 promoter, wherein the functional fragment of m3′HS1 insulator preferably comprises or consists of a nucleotide sequence having a CTCF motif and at least 90% preferably at least 95, 96, 97, 98 or 99% identity to the full length sequence as set forth in SEQ ID NO:4, and retaining an insulator activity.

In another embodiment, the multivalent rHVT comprises in a first insertion site located in the non-coding region between UL45 and UL46, a first recombinant nucleotide sequence encoding a VP2 protein of IBDV or an antigenic fragment thereof operably linked to a promoter, preferably a Bac promoter, and a m3′HS1 insulator, or a functional fragment thereof, positioned upstream of the Bac promoter and, in a second insertion site located in the non-coding region between SORF3 and US2, a second recombinant nucleotide sequence encoding a gB protein of ILTV or an antigenic fragment thereof operably linked to a promoter, preferably a Mcmv ie1 promoter, wherein the functional fragment of m3′HS1 insulator preferably comprises or consists of a nucleotide sequence having a CTCF motif and at least 90%, preferably at least 95, 96, 97, 98 or 99% identity to the full length sequence as set forth in SEQ ID NO:4, and retaining an insulator activity.

In another embodiment, the multivalent rHVT comprises in a first insertion site located in the non-coding region between UL44 and UL45, a first recombinant nucleotide sequence encoding a VP2 protein of IBDV or an antigenic fragment thereof operably linked to a promoter, preferably a Mcmv ie1 promoter, and a m3′HS1 insulator, or a functional fragment thereof, positioned upstream of the Mcmv ie1 promoter and, in a the second insertion site located in the non-coding region between UL45 and UL46, a second recombinant nucleotide sequence encoding a gB protein of ILTV or an antigenic fragment thereof operably linked to a promoter, preferably a Pec promoter, wherein the functional fragment of m3′HS1 insulator preferably comprises or consists of a nucleotide sequence having a CTCF motif and at least 90%, preferably at least 95, 96, 97, 98 or 99% identity to the full length sequence as set forth in SEQ ID NO:4, and retaining an insulator activity.

In still another embodiment, the multivalent rHVT comprises in a first insertion site located in the non-coding region between SORF3 and US2, a first recombinant nucleotide sequence encoding a VP2 protein of IBDV or an antigenic fragment thereof operably linked to a promoter, preferably a Mcmv ie1 promoter, and a m3′HS1 insulator, or a functional fragment thereof, positioned upstream of the Mcmv ie1 promoter and, in a second insertion site located in the non-coding region between UL45 and UL46, a second recombinant nucleotide sequence encoding a gB protein of ILTV or an antigenic fragment thereof operably linked to a promoter, preferably a Pec promoter, wherein the functional fragment of m3′HS1 insulator preferably comprises or consists of a nucleotide sequence having a CTCF motif and at least 90%, preferably at least 95, 96, 97, 98 or 99% identity to the full length sequence as set forth in SEQ ID NO:4, and retaining an insulator activity.

It is an object of the present invention to provide a rHVT comprising a first nucleotide sequence encoding the VP2 protein of IBDV, preferably of SEQ ID NO:1 or having at least 80, 85, 95, 96, 97, 98 or 99% identity to the full length sequence as set forth in SEQ ID NO:1, located under the control of Bac promoter, preferably of SEQ ID NO:2 or having at least 80, 85, 90, 95, 96, 97, 98 or 99% identity to the full length sequence as set forth in SEQ ID NO, flanked with the insulator sequence of SEQ ID NO:4 or having at least 90, 95, 96, 97, 98 or 99% identity to the full length sequence as set forth in SEQ ID NO:4, at the 5′-end and SV40 poly adenylation signal, preferably of SEQ ID NO:3 or having at least 80, 85, 90, 95, 96, 97, 98 or 99% identity to the full length sequence as set forth in SEQ ID NO:3, at the 3′-end, inserted into the first intergenic region between HVT053 (UL45) and HVT054 (UL46), and a second nucleotide sequence encoding the gB protein of ILTV, preferably of SEQ ID NO:5 or having at least 80, 85, 90, 95, 96, 97, 98 or 99% identity to the full length sequence as set forth in SEQ ID NO:5, located under the control of Mcmv ie1 promoter, preferably of SEQ ID NO: 6 or having at least 80, 85, 90, 95, 96, 97, 98 or 99% identity to the full length sequence as set forth in SEQ ID NO:6, linked to poly adenylation signal-1, preferably of SEQ ID NO:7 or having at least 80, 85, 90, 95, 96, 97, 98 or 99% identity to the full length sequence as set forth in SEQ ID NO:7, at the 3′-end, inserted into a second intergenic region between HVT052 (UL44) and HVT053 (UL45) (FW311).

It is another object of the present invention to provide a rHVT comprising a first nucleotide sequence encoding the VP2 protein of IBDV, preferably of SEQ ID NO:1, located under the control of Mcmv ie1 promoter, preferably of SEQ ID NO:6 or having at least 80, 85, 90, 95, 96, 97, 98 or 99% identity to the full length sequence as set forth in SEQ ID NO:6, flanked with the insulator sequence of SEQ ID NO:4 or having at least 90, 95, 96, 97, 98 or 99% identity to the full length sequence as set forth in SEQ ID NO:4, at the 5′-end and SV40 poly adenylation signal, preferably of SEQ ID NO:3 or having at least 80, 85, 90, 95, 96, 97, 98 or 99% identity to the full length sequence as set forth in SEQ ID NO:3, at the 3′-end, inserted into a first intergenic region between HVT052 (UL44) and HVT053 (UL45), and a second nucleotide sequence encoding the gB protein of ILTV, preferably of SEQ ID NO:5 or having at least 80, 85, 90, 95, 96, 97, 98 or 99% identity to the full length sequence as set forth in SEQ ID NO:5, located under the control of Pec promoter, preferably of SEQ ID NO:18 or having at least 80, 85, 90, 95, 96, 97, 98 or 99% identity to the full length sequence as set forth in SEQ ID NO:18, linked to poly adenylation signal-1, preferably of SEQ ID NO:7 or having at least 80, 85, 90, 95, 96, 97, 98 or 99% identity to the full length sequence as set forth in SEQ ID NO:7, at the 3′-end, inserted into a second intergenic region between HVT053 (UL45) and HVT054 (UL46) (FW313).

### Virus Construction

The recombinant MDV, preferably recombinant HVT, of the invention may be prepared using techniques known per se in the art, such as recombinant technology, homologous recombination, site-specific insertion, mutagenesis, and the like.

Gene cloning and plasmid construction are well known to one person of ordinary skill in the art and may be essentially performed by standard molecular biology techniques (Molecular Cloning: A Laboratory Manual. 4^{th} Edition, Cold Spring Harbor Laboratory, Cold Spring Harbor, New York, USA, 2012).

Typically, the recombinant viruses may be prepared by homologous recombination between the viral genome and a construct (e.g., a plasmid) comprising the nucleic acid to be inserted, flanked by nucleotides from the insertion site to allow recombination. Insertion can be made with or without deletion of endogenous sequences.

The resulting recombinant virus may be selected genotypically or phenotypically using known techniques of selection, e.g., by hybridization, detecting enzyme activity encoded by a gene integrated along with the recombinant nucleic acid sequences or detecting the antigenic peptide expressed by the recombinant virus immunologically. The selected recombinant virus can be cultured on a large scale in cell culture after which, recombinant virus containing peptides can be collected.

### Cell Cultures

The recombinant viruses of the present invention may be propagated in any competent cell cultures. After required growth of the viruses is achieved, the cells may be detached from the wells using a scraper or with trypsin and the infected cells may be separated from the supernatant by centrifugation.

Examples of competent cells include CEF, embryonated egg, chicken kidney cell, and the like. The cells or viruses may be cultured in a culture medium such as Eagle's MEM, Leibowitz-L-15/McCoy 5A (1:1 mixture) culture medium at about 37° C. for 3 to 6 days. The infected cells are typically suspended in a culture medium containing 10% dimethyl sulfoxide (DMSO) or CELLBANKER® 1 (ZENOAQ) and stored frozen under liquid nitrogen or in a deep freezer at e.g., −85° C.

The invention also relates to a method for producing or replicating a rMDV preferably a rHVT, as described above, comprising infecting a competent host cell with the rMDV or a nucleic acid molecule comprising, consisting essentially of, or consisting of the genome of the rMDV, preferably the genome of the rHVT, and collecting the rMDV, preferably the rHVT.

The invention also relates to a host cell comprising a rMDV, preferably a rHVT, as described above or a nucleic acid molecule comprising, consisting essentially of, or consisting of the genome of a rMDV, preferably of the genome of a rHVT.

Advantageously, the rMDV of the invention present a high level of stability through passages, which corresponds to an expression of the recombinant nucleotide sequence(s) in cells of avian species even after 10, 15, 20 or more passages. In the context of the invention a “passage” or “cell passaging” means a culture of cells in suitable conditions for allowing their growth and keeping them alive until they are 90% to 100% confluent. The passaging step consists on transferring a small number of cells of the previous confluent culture into a new culture medium. An aliquot of the previous confluent culture, containing a few cells, may be diluted in a large volume of fresh medium. In case of adherent cultures, cells may be first detached, for example by using a mixture of trypsin and EDTA, or any suitable enzyme, before to use a few number of detached cells for seeding a new culture medium.

According to preferred embodiments of the invention, CEF cells transfected with rMDV of the invention still express the corresponding antigen(s) after at least 10 passages. In other words, CEF cells resulting from 10 or more passages of CEF cells transfected with rMDV of the invention, and more particularly resulting from 15 passages, still contain the foreign nucleotide sequence(s) of the rMDV used for the initial cell transfection and express the corresponding antigen(s). In the context of the invention, one considers that cells of a said passage still express the antigen(s) if the level of production is greater than 80% of the level of production of the first passage, and preferentially greater than 85%.

### Compositions, Vaccines and Uses Thereof

The invention also relates to compositions, including vaccines, which comprise an effective immunizing amount of a monovalent or a multivalent recombinant MDV, preferably recombinant HVT, of the invention, a nucleic acid of the invention, or a cell of the invention. By “effective immunizing amount” is meant amount of rMDV, preferably rHVT, of the invention which is sufficient to produce an immunological response. An effective amount may vary with the antigen(s). The quantity which constitutes an effective amount may vary depending on whether the vaccine is intended as a first treatment or as a booster treatment.

Vaccines of the invention typically comprise an immunologically effective amount of a recombinant MDV, preferably recombinant HVT, as described above, in a pharmaceutically acceptable vehicle.

The compositions and vaccines according to the present invention typically comprise a suitable solvent or diluent or excipient, such as for example an aqueous buffer or a phosphate buffer. The compositions may also comprise additives, such as proteins or peptides derived from animals (e.g., hormones, cytokines, co-stimulatory factors), nucleic acids derived from viruses and other sources (e.g., double stranded RNA, CpG), and the like which are administered with the vaccine in an amount sufficient to enhance the immune response. In addition, any number of combinations of the aforementioned substances may provide an immunopotentiation effect, and therefore, can form an immunopotentiator of the present invention.

The vaccines of the present invention may further be formulated with one or more further additives to maintain isotonicity, physiological pH and stability, for example, a buffer such as physiological saline (0.85%), phosphate-buffered saline (PBS), citrate buffers, Tris hydroxymethyl aminomethane (TRIS), Tris-buffered saline and the like, or an antibiotic, for example, neomycin or streptomycin, etc.

The rMDV according to the invention may be preferably used as a live vaccine, although other alternatives like inactivated vaccines or attenuated vaccines are well within the skill of a person skilled in the art.

The route of administration can be any route including oral (e.g., drinking water, gel), ocular (e.g., by eyedrop), oculo-nasal administration using aerosol (e.g by spray), intranasal, cloacal, in ovo, topically, or by injection (e.g., intravenous, subcutaneous, intramuscular, intraorbital, intraocular, intradermal, and/or intraperitoneal) vaccination. The skilled person will easily adapt the formulation of the vaccine composition for each type of route of administration.

Each vaccine dose may contain a suitable dose sufficient to elicit a protective immune response in avian species. Optimization of such dose is well known in the art. The amount of antigen per dose may be determined by known methods using antigen/anti-body reactions, for example by the ELISA method.

The vaccines of the invention can be administered as single doses or in repeated doses, depending on the vaccination protocol.

The vaccines of the present invention are further advantageous in that they confer to bird species up to 70% protection against the targeted avian pathogens after 4 weeks of vaccination, preferably up to 80%, 90% or more.

The present invention further relates to the use of the composition, vaccine or vaccine composition as described above for immunizing or vaccinating avian species, such as poultry, against at least one pathogen.

The present invention further relates to a rMDV, preferably a rHVT, as described above for use for immunizing or vaccinating an avian such as a poultry, preferably a chicken, against at least one avian pathogen.

The present invention further relates to a method of immunizing or vaccinating avian species by administering an immunologically effective amount of the vaccine according to the invention. The vaccine may be advantageously administered intradermally, subcutaneously, intramuscularly, orally, in ovo, by mucosal administration or via oculo-nasal administration.

The present invention further relates to a rMDV as described above for use for increasing the onset of immunity in an avian such as a poultry, preferably a chicken, against at least one avian pathogen. By “increasing” the onset of immunity is meant providing to the avian an immune protection against a pathogen stronger after vaccination with rMDV, preferably rHVT, as herein described, than when using vaccines not comprising insulator element.

In a particular embodiment, the rMDV or vaccine composition is for use for vaccinating an avian such as a poultry, preferably a chicken, against Newcastle disease virus (NDV).

In another embodiment, the rMDV or vaccine composition is for use for vaccinating an avian such as a poultry, preferably a chicken, against both infectious bursal disease virus (IBDV) and Infectious laryngotracheitis virus (ILVT).

In another embodiment, the rMDV or vaccine composition is for use for vaccinating an avian such as a poultry, preferably a chicken, against infectious bursal disease virus (IBDV).

The present invention further relates to vaccination kits for immunizing avian species which comprises an effective amount of the monovalent or multivalent vaccine as described above and means for administering said components to said species. For example, such kit comprises an injection device filled with the monovalent or multivalent vaccine according to the invention and instructions for intradermic, subcutaneous, intramuscular, or in ovo injection. Alternatively, the kit comprises a spray/aerosol, gel drop or eye drop device filled with the multivalent vaccine according to the invention and instructions for oculo-nasal administration, oral or mucosal administration.

Further aspects and advantages of the present application will now be disclosed in the following examples, which are illustrative of the invention.

### EXAMPLES

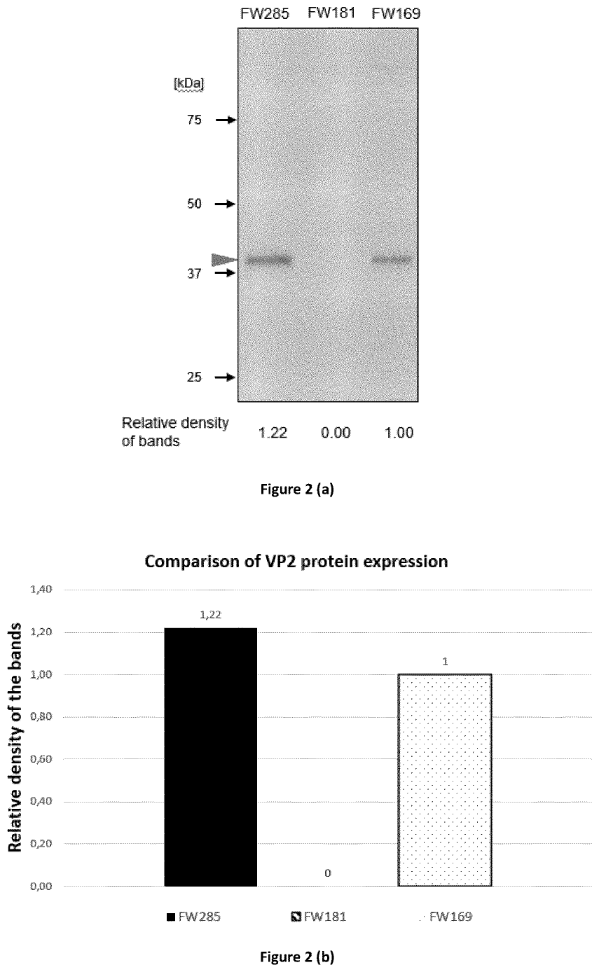

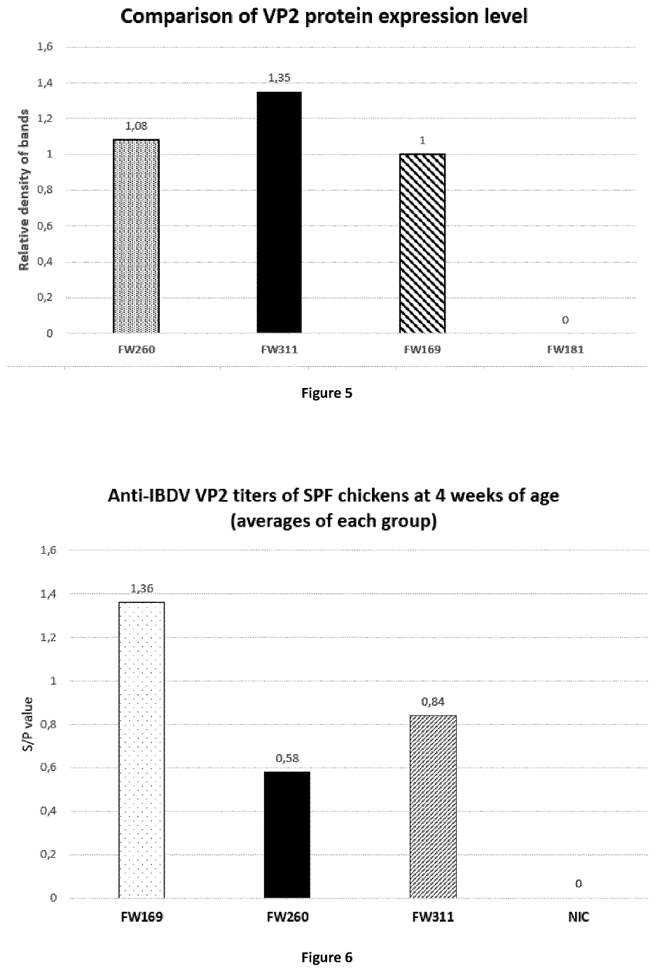

The inventors constructed a series of recombinant HVT with different expression cassettes inserted in the non-coding regions located between HVT053 (UL45) and HVT054 (UL46), or HVT052 (UL44) and HVT053 (UL45), or HVT087 (SORF3) and HVT088 (US2). Schematic diagrams of them are shown in FIG. 2(a) and FIG. 5(a).

In the experiments, several monovalent and multivalent recombinant HVT have been used in each efficacy trial. These viruses are designated as follows (virus/insertion site_inserted expression cassette):

FW169: rHVT/HVT053-054_Bac-VP2 (vaccine control)
FW181: rHVT/HVT053-054_Pec-gBdel
FW285: rHVT/HVT053-054_m3′HS1-72-Bac-VP2
FW348: rHVT/HVT053-054_m3′HS1-72-Bac-F
FW260: rHVT/HVT053-054_Bac-VP2/HVT052-053_Mcmvie1-gBdel
FW311: rHVT/HVT053-054_m3′HS1-72-Bac-VP2/HVT052-053_Mcmvie1-gBdel
FW313: rHVT/HVT052-053_m3′HS1-72-Mcmvie1-VP2/HVT053-054_Pec-gBdel

### Example 1: Construction of Homology Vectors

The plasmid construction was essentially performed by the standard molecular biology techniques (Molecular Cloning: A Laboratory Manual. 4^{th} Edition, Cold Spring Harbor Laboratory, Cold Spring Harbor, New York, USA, 2012).

Construction of p45/46 m3′HS1-72 Bac-VP2

The fragment where the insulator sequence, m3′HS1-72 (SEQ ID NO:4), was directly linked to the 5′-terminus of Bac promoter was made by overlapping PCR with p45/46BacVP2 (wo03064595) and the specific primers (SEQ ID NO: 9, 10, 11 and 12). That amplicon was cloned into XbaI and EcoRI-digested p45/46BacVP2, resulting in p45/46_m3′HS1-72-Bac-VP2.

Construction of p44/45d46 Mcmvie1-gBdel

The nucleotide sequence of ILTV-gBdel gene (SEQ ID:5) was originally obtained by cloning from ILTV strain 632 (EP1731612A1). The ILTV_gBdel gene was cloned into pUC18 plasmid so that ILTV_gBdel gene has an additional XbaI site and SalI site at 5′-terminus and 3′-terminus respectively. Then, the portion of ILTV_gBdel was divided to two pieces by digestion with XbaI and SalI. Those two pieces of ILTV_gBdel gene were cloned into XbaI and SalI-digested p44/45d46_Mcmvie1-VP2 (WO13144355), resulting in p44/45d46_Mcmvie1-gBdel.

Construction of p45/46 Bac-F

NDV-F gene was synthesized and replaced with IBDV-VP2 gene of p45/46BacVP2 (U.S. Pat. No. 7,153,511) by digestion with XbaI and SalI, resulting in p45/46_Bac-F.

Construction of p45/46 m3′HS1-72 Bac-F

The fragment containing the insulator sequence, m3′HS1-72 (SEQ ID NO:4), was digested from p45/46_m3′HS1-72_Bac-VP2 with XhoI. That fragment was cloned into XhoI-digested p45/46_Bac-F, resulting in p45/46_m3′HS1-72_Bac-F.

Construction of p44/45d46 m3′HS1-72 Mcmvie1-VP2

The fragment of Mcmvie1 promoter linked with the insulator sequence (SEQ ID:4) at 5′-terminus was amplified by overlapping PCR with p44/45d46 Mcmvie1-VP2 (wo13144355) and the specific primers (SEQ ID:19, 20, 21, 22, 23, and 24). That fragment was inserted into SacII and NaeI-digested p44/45d46_Mcmvie1-VP2 by In-Fusion cloning, resulting in p44/45d46_m3′HS1-72-Mcmvie1-VP2.

### Example 2: Construction of Recombinant HVT

Construction of recombinant HVT was conducted by homologous recombination in cultured cells. For homologous recombination in cultured cells, viral DNAs of wild type HVT FC-126 strain (PASS+14) and FW285 (PASS+25) and FW181 (PASS+22) were prepared as described by Morgan et al. (Avian Diseases, 34:345-351, 1990). Approximately 2 μg of the parent viral DNA and 1 μg of one of the homology vector were transfected into approximately 10⁷ CEF cells by electroporation using Nucleofector II (Lonza, Basel, Switzerland). The transfected cells were added to Leibovitz's L-15 (Life Technologies Corp., Cat. #41300-39), McCoy's 5A Medium (Life Technologies Corp., Cat. #21500-061) (1:1) and 4% calf serum [LM (+) medium], planted in 96-well tissue culture plates, and then incubated at 37° C. in 4-5% CO₂ for 5-7 days until recombinant HVT plaques became visible. The cells were then detached from the plates by trypsinization, transferred equally to two 96-well plates with CEF, and incubated for 4 to 6 days until plaques were observed. Screening was conducted by the black plaque assay, staining only plaques expressing IBDV VP2 protein or ILTV gB protein. Briefly, one of the two plates was fixed with methanol:acetone mixture (1:2) and incubated with anti-IBDV VP2 mouse monoclonal antibody R63 (ATCC #: HB-9490) or anti-ILTV gB mouse monoclonal antibody #1_B4_7 (unpublished). Next, incubated with biotinylated anti-mouse IgG antibody (Vector Laboratories, Cat #BA-9200) and then with VECTASTAIN ABC-AP kit (Vector Laboratories, Cat #AK-5000), plaques expressing VP2 protein or gB protein were stained by addition of NBT/BCIP solution (Roche Applied Science, Cat #1681451). Wells containing stained recombinant plaques were identified and cells from the corresponding wells on the other 96-well plate were trypsinized. The cells were then diluted in fresh secondary CEF cells and transferred to 96-well plates to complete the first round of purification. The purification procedure was repeated until all plaques were stained positively in the black plaque assay.

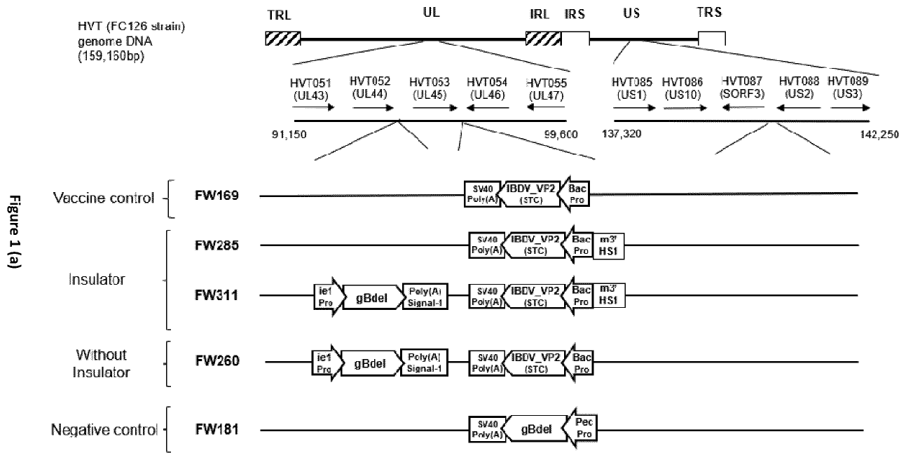

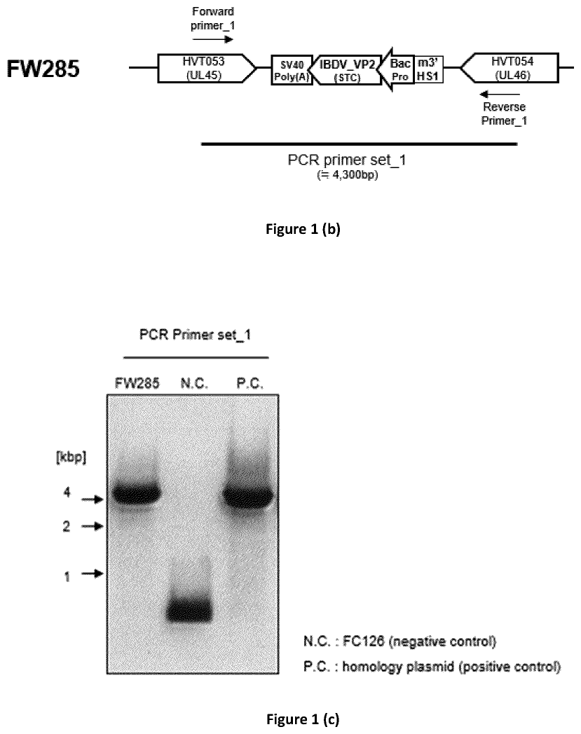

A list of the constructed recombinant HVTs, their parent viruses and the homology vectors used is provided in Table I below. Diagrams showing genomic structures of the recombinant HVT/IBDs and recombinant HVT/IBD-LTs are provided in FIG. 1(a).

TABLE I
constructed recombinant HVT, parent
viruses, and homology vectors

| Construct code | Parent virus | Homology vector |
|---|---|---|
| FW169 (monovalent | HVT FC-126 strain | p45/46_Bac-VP2 |
| control) | | |
| FW181 (monovalent | HVT FC-126 strain | p45/46_Pec-gBdel |
| control) | | |
| FW026 (monovalent) | HVT FC-126 strain | P45/46_Bac-F |
| FW285 (monovalent) | HVT FC-126 strain | p45/46_m3′HS1-72- |
| | | Bac-VP2 |
| FW348 (monovalent) | HVT FC-126 strain | P45/46_m3′HS1-72_Bac-F |
| FW260 (bivalent | FW169 | p44/45d46_Mcmvie1- |
| control) | | gBdel |
| FW311 (bivalent) | FW285 | p44/45d46_Mcmvie1- |
| | | gBdel |
| FW313 (bivalent) | FW181 | P44/45d46_m3′HS1- |
| | | 72_Mcmvie1-VP2 |

Construction of FW169 and FW181 was performed as disclosed in WO03064595.

### Example 3: Verification of Genome Structure of Recombinant HVT/IBDs

With FW285 as a model case, characterization method of recombinant HVT/IBD is described below. Briefly, genome structures of the recombinant HVT/IBDs were verified by PCR reaction amplifying flanking region of the inserted genes. FIG. 1(b) shows where is the amplified region in FW285. The primer pairs used in the PCR reaction are SEQ ID NO:13 and SEQ ID NO:14. FIG. 1(c) demonstrates that all clones of FW285 have the correct genome structure and those clones are free from the parent virus.

### Example 4: Comparison of VP2 Protein Expression from Recombinant HVT/IBDs

Expression of a VP2 antigen by the recombinant HVT/IBD of the invention (FW285) was confirmed by the Western blot assay. The western blot was conducted using CEF cells infected with the recombinant viruses and anti-IBDV VP2 mouse monoclonal antibody R63. Briefly, CEF cells in 12-well plates were infected with one of the recombinant viruses or the other recombinant virus strain at a multiplicity of infection of approximately 0.1. Three days post inoculation, cells were harvested with trypsin and centrifuged at 913×g for 5 minutes. The pellet was washed with PBS and resuspended with 100 μl of PBS. After adding the same volume of 2×SDS sample buffer (130 mM Tris-Cl (pH6.8), 6% SDS, 20% Glycerol, 10% 2-Mercaptoethanol and 0.01% Bromo Phenol Blue), cell suspension was boiled for 5 minutes. The samples were separated by SDS-PAGE using 10% polyacrylamide gel and transferred to a PVDF membrane (Immobilon-P, Millipore). The membrane was dried completely and then incubated with anti-IBDV VP2 mouse monoclonal antibody R63. After anti-IBDV VP2 mouse monoclonal antibody R63 was washed off, biotinylated anti-mouse IgG antibody (Vector Laboratories, Cat #BA-9200) and then with VECTASTAIN ABC-AP kit (Vector Laboratories, Cat #AK-5000). Protein bound with the anti-IBDV VP2 mouse monoclonal antibody R63 was visualized by addition of NBT/BCIP solution (Roche Applied Science, Cat #1681451).

As shown in FIG. 2(a), protein bands of 40 kilodaltons (kDa), which was the expected size of the VP2 protein, were observed in the lanes with rHVT/IBD infected cells (FW285 and FW169). The in vitro results clearly demonstrate that the inclusion of a single upstream insulator does not alter the promoter transcriptional activity.

To compare the amount of expressed VP2 protein among recombinant HVT/IBDs, the result of western blot assay was quantified by ImageJ and shown as a bar graph in FIG. 2(b). The relative densities of bands were measured with respect to the vaccine control, FW169. It was demonstrated that insulator sequence can enhance the VP2 protein expression compared with rHVT without insulator in vitro.

### Example 5: Efficacy of Recombinant HVT/IBDs in SPF Chickens

The efficacy of FW285 was further investigated in SPF chickens. Chickens at one day of age were divided into four groups and chicks in Groups 3 and 4 were vaccinated subcutaneously with approximately 3000 plaque forming units (pfu)/0.2 ml of one of the recombinant HVT/IBD (FW169: Group 3, FW285: Group 4). Chicks in Group 2 (non-immunized, challenged positive control—NIC) were left unvaccinated. Chicks in Group 1 (non-immunized, non-challenged control—NINC) were left unvaccinated and unchallenged. The chickens were bled each week between 1 and 4 weeks of age and tested for presence of anti-IBDV antibodies with a commercial IBDV ELISA kit (ID Screen® IBD VP2: Idvet). Challenge was conducted at 4 weeks of age. For challenge, 1×10³ EID₅₀ of virulent IBDV STC strain was administered via oral route. Chickens were observed daily for clinical signs associated with IBD, such as depression and death. Seven days post challenge, chickens were necropsied and observed for grossly observable bursal lesions such as edema, discoloration, atrophy, hemorrhage, and yellow or gelatinous exudates. Weights of body and bursa were also measured at necropsy for calculation of B/B index, which is the ratio between the weight of the bursa and the body weight of challenged birds divided by the same ratio of non-challenged birds.

The results of IBDV ELISA are shown in FIG. 3, and the results of efficacy are summarized Table II below.

Construct FW285 appeared to induce the production of anti-VP2 antibodies in vaccinated chicken earlier than in chickens vaccinated with the control FW169 vaccine (OOI at 2 weeks for FW285, and OOI at 3 weeks for FW169), confirming an early onset of immunity with the construct of the present invention. Moreover, a higher anti-IBDV VP2 titer is obtained with FW285 construct as early as 3 weeks and up to 4 weeks after vaccination, as compared to the anti-IBDV VP2 titer obtained with FW169 vaccine, confirming a stronger immunity with the construct of the present invention.

TABLE II
Protection of recombinant HVT/IBD against virulent
IBDV challenge at 4 weeks of age in SPF chickens

| | | | | | Number of | |
|---|---|---|---|---|---|---|
| | | | | | chickens | |
| | | | | Number of | with bursal | |
| | | Number | B/B | chickens | lesions/total | % |
| | | of | Index | dead after | number of | protection |
| Group | Group | chickens | (*) | challenge | chickens | (**) |
| 1 | NINC | 15 | 1.00 | 0 | 0 | 100 |
| 2 | NIC | 15 | 0.51 | 6 | 9 | 0 |
| 3 | FW169 | 15 | 1.02 | 0 | 2 | 87 |
| | (vaccine | | | | | |
| | control) | | | | | |
| 4 | FW285 | 15 | 1.08 | 0 | 2 | 87 |

(*) B/B index means Bursal-Body index
(**) % protection = 100-100 × (# dead + # lesion)/nB/B index is calculated as follow:
BB index = BB ratio of infected (or vaccinated) birds/BB ratio of the controls
wherein BB ratio = [bursa weight (g)/body weight (g)] × 1000

As indicated in the publication “Bursal body index as a visual indicator for the assessment of bursa of Fabricius” (Journal of Veterinary Medicine and Animal Health vol. 9(2), pp 32-38, February 2017; DOI: 10.5897/JVMAH2016.0456), an index below 0.7 is usually considered indicative of bursal atrophy, in contrast, a B/B index above value 0.7 is indicative of no bursal atrophy.

Table II confirms that FW285 is stable in vivo. Table II further shows that FW285 can effectively protect chickens from IBDV challenge as well as FW169. Furthermore, FW285 elicits better anti-IBDV VP2 antibody titer than FW169 (FIG. 3).

These data indicate that insulator sequence can enhance both the transgene expression and antibody titer of rHVT in vivo. An earlier animal's onset of immunity may be further obtained when avian are vaccinated with rHVT of the invention.

### Example 6: Verification of Genome Structure of Recombinant HVT/NDs

With FW348 as a model case, characterization method of recombinant HVT/ND is described below. Briefly, genome structures of the recombinant HVT/NDs were verified by PCR reaction amplifying flanking region of the inserted genes. The primer pairs used in the PCR reaction are SEQ ID NO:25 and SEQ ID NO:26.

### Example 7: Comparison of F Protein Expression from Recombinant HVT/NDs

Expression of a F antigen by the recombinant HVT/ND of the invention (FW348) was confirmed by the Black plaque assay and Western blot assay. The Black plaque and western blot were conducted using CEF cells infected with the recombinant viruses and anti-NDV F mouse monoclonal antibody #77-2.

For Black plaque, CEF cells in 12-well plates were infected with one of the recombinant viruses at a multiplicity of infection of approximately 0.1. Three days post inoculation, cells were fixed by methanol:acetone mixture (1:2). After 3 times washing by PBS, the samples were incubated with anti-NDV F mouse monoclonal antibody #77-2. After anti-NDV F mouse monoclonal antibody #77-2 was washed off, the samples were incubated with biotinylated anti-mouse IgG antibody (Vector Laboratories, Cat #BA-9200), and then with VECTASTAIN ABC-AP kit (Vector Laboratories, Cat #AK-5000). Finally, NDV-F protein expression was visualized by NBT/BCIP solution (Roche Applied Science, Cat #1681451). The result was observed by using a microscope.

For Western blot, CEF cells in 12-well plates were infected with one of the recombinant viruses or the other recombinant virus strain at a multiplicity of infection of approximately 0.1. Three days post inoculation, cells were harvested with trypsin and centrifuged at 913×g for 5 minutes. The pellet was washed with PBS and resuspended with 100 μl of PBS. After adding the same volume of 2×SDS sample buffer (130 mM Tris-Cl (pH6.8), 6% SDS, 20% Glycerol, 10% 2-Mercaptoethanol and 0.01% Bromo Phenol Blue), cell suspension was boiled for 5 minutes. The samples were separated by SDS-PAGE using 10% polyacrylamide gel and transferred to a PVDF membrane (Immobilon-P, Millipore). The membrane was dried completely and then incubated with anti-NDV F mouse monoclonal antibody #77-2. After anti-NDV F mouse monoclonal antibody #77-2 was washed off, biotinylated anti-mouse IgG antibody (Vector Laboratories, Cat #BA-9200) and then with VECTASTAIN ABC-AP kit (Vector Laboratories, Cat #AK-5000). Protein bound with the anti-NDV F mouse monoclonal antibody #77-2 was visualized by addition of NBT/BCIP solution (Roche Applied Science, Cat #1681451).

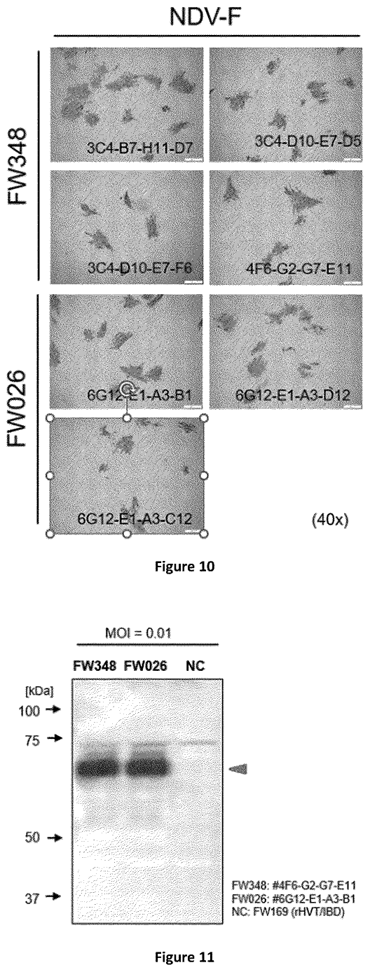

As shown in FIG. 10, NDV F protein expression was observed in CEF cells infected with rHVT/NDs.

As shown in FIG. 11, protein bands of 60 kilodaltons (kDa), which was the expected size of the F protein, were observed in the lanes with rHVT/ND infected cells (FW348 and FW026). The in vitro results clearly demonstrate that the inclusion of a single upstream insulator does not alter the promoter transcriptional activity.

### Example 8: Verification of Genome Structure of Recombinant HVT/IBD-LTs

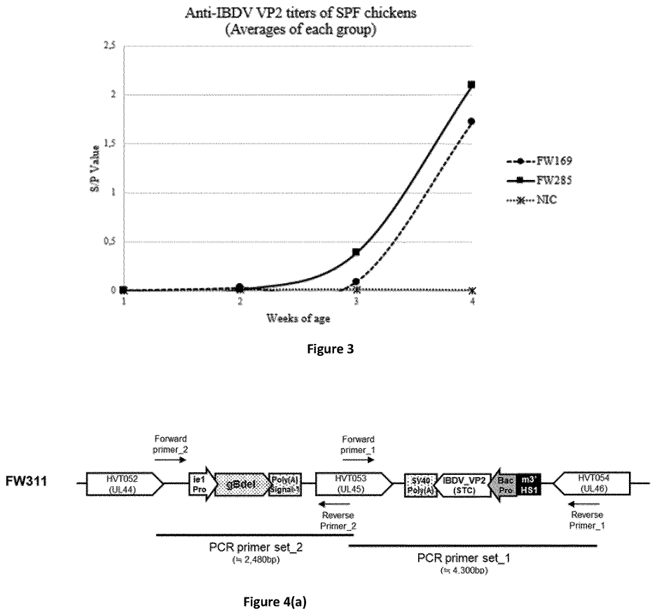

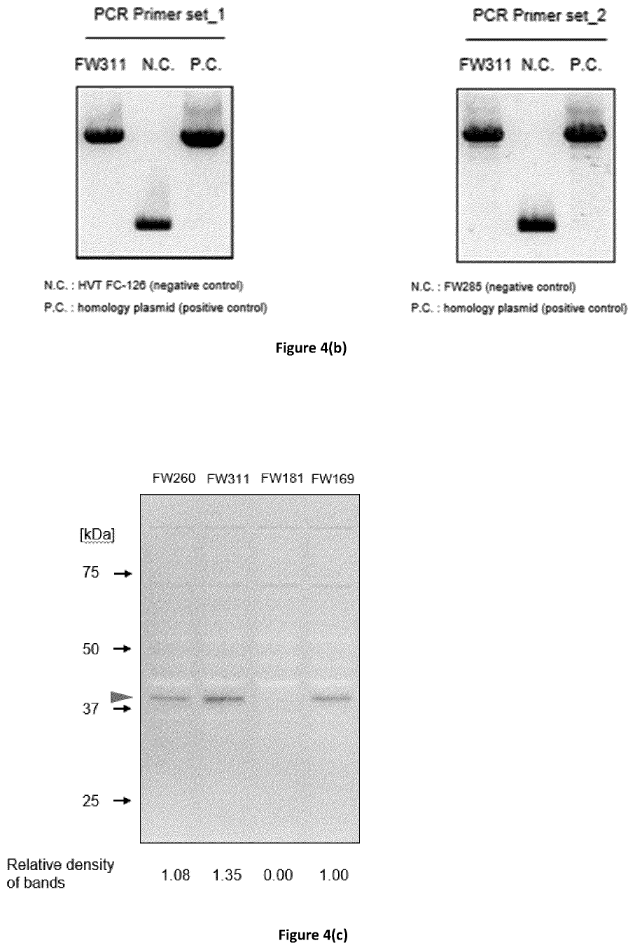

With FW311 as a model case, characterization method of recombinant HVT/IBD-LT is described below. Briefly, genome structures of the recombinant HVT/IBD-LTs were verified by PCR reactions amplifying flanking regions of the inserted genes. FIG. 4(a) shows the amplified regions in FW311. The primer pairs used in the PCR reaction are SEQ ID NO:13 and SEQ ID NO:14 for PCR primer set 1, SEQ ID NO:15 and SEQ ID NO:16 for PCR primer set_2. FIG. 4(b) demonstrates that FW311 has the correct genome structure and is free from the parent virus.

### Example 9: Comparison of VP2 Protein Expression from Recombinant HVT/IBD-LTs

Expression of a VP2 antigen by recombinant HVT/IBD-LT of the invention (FW311, 313) was confirmed by Western blot assay. The western blot were conducted using CEF cells infected with the recombinant viruses and anti-IBDV VP2 mouse monoclonal antibody R63, as exposed in Example 4.

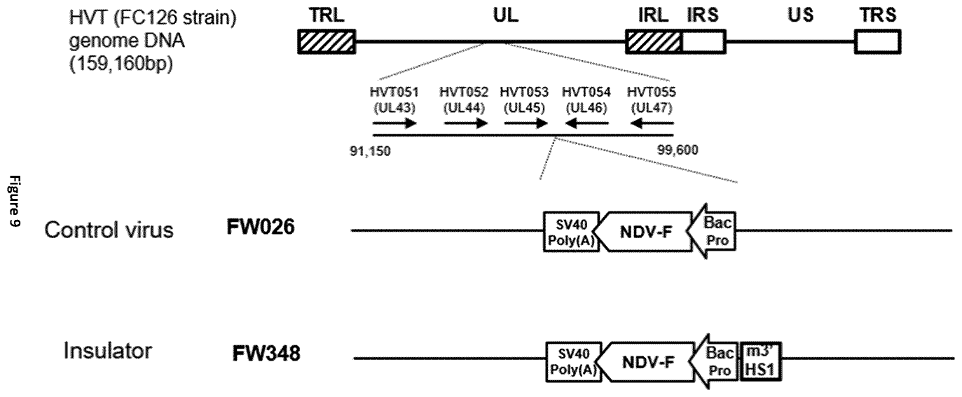

Protein bands of 40 kilodaltons (kDa), which was the expected size of the VP2 protein, were observed in the lanes with rHVT/IBD-LTs infected cells. FW260, is multivalent rHVT lacking an insulator and was used as counter-parts of FW311 (FIG. 4(c)). Similarly, FW261 is multivalent rHVT lacking an insulator and was used as counter-parts of FW313 (FIG. 8(a)).

To compare the amount of expressed VP2 protein among recombinant HVT/IBD-LTs, the result of western blot assay was quantified by ImageJ and shown as a bar graph in FIG. 5 (FW311) and FIG. 8b (FW313). The relative densities of bands were measured with respect to the vaccine control, FW169 (FIG. 5). It was demonstrated that insulator sequence can enhance the VP2 protein expression compared with rHVT without insulator in vitro.

### Example 10: Efficacy of Recombinant HVT/IBD-LTs Against Virulent IBDV in SPF Chickens (FW311)

The efficacy of recombinant HVT/IBD-LTs FW311was investigated in SPF chickens. Chickens at one day of age were divided into four groups and chicks of Group 4 were vaccinated subcutaneously with approximately 3000 plaque forming units (pfu)/0.2 ml of the recombinant HVT/IBD-LT (FW311: Group 4,). Similarly, chicks in Group 3 were vaccinated subcutaneously with vaccine control (FW169. Chicks in Group 2 (non-immunized, challenged positive control) were left unvaccinated. Chicks in Group 1 (non-immunized, non-challenged control) were left unvaccinated and unchallenged. The chickens were bled each week between 1 and 4 weeks of age. Challenge was conducted at 4 weeks of age. For challenge, 1×10³ EID₅₀ of virulent IBDV STC strain was administered via oral route. Chickens were observed daily for clinical signs associated with IBD, such as depression and death. Seven days post challenge, chickens were necropsied and observed for grossly observable bursal lesions such as edema, discoloration, atrophy, hemorrhage, and yellow or gelatinous exudates. Weights of body and bursa were also measured at necropsy for calculation of B/B index, which is the ratio between the weight of the bursa and the body weight of challenged birds divided by the same ratio of non-challenged birds.

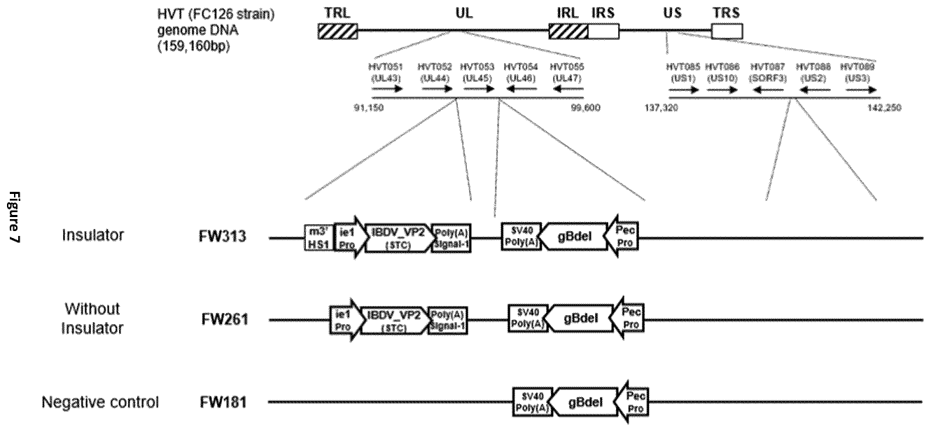

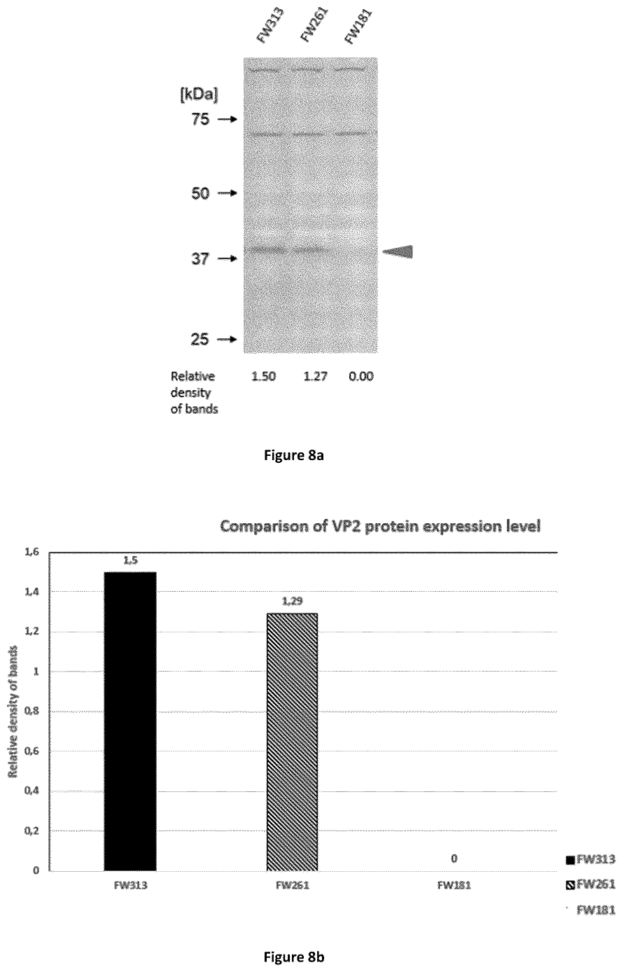

The results of IBDV ELISA are shown in FIG. 6, and the results of Efficacy trial are summarized Table III below.

Construct FW311 induced the synthesis of anti-VP2 antibodies.

TABLE III
Protection of recombinant HVT/IBD-LT against virulent IBDV challenge
at 4 weeks of age in SPF chickens (Efficacy trial 2)

| | | | | | Number of | |
|---|---|---|---|---|---|---|
| | | | | | chickens | |
| | | | | Number of | with bursal | |
| | | Number | B/B | chickens | lesions/total | % |
| | | of | Index | dead after | Number of | protection |
| Group | Group | chickens | (*) | challenge | chickens | (**) |
| 1 | NINC | 15 | 1.00 | 0 | 0 | 100 |
| 2 | NIC | 15 | 0.56 | 10 | 5 | 0 |
| 3 | FW169 | 15 | 0.91 | 0 | 0 | 100 |
| | (vaccine | | | | | |
| | control) | | | | | |
| 4 | FW311 | 15 | 0.88 | 1 | 2 | 80 |

(*) B/B index means Bursal-Body index
(**) % protection = 100-100 × (# dead + # lesion)/n.

The results confirm that FW311 is stable in vivo. The results further show that FW311 elicits a protective immunity against IBDV challenge infection in vivo.

### Example 11: Efficacy of Recombinant HVT/IBD-LTs Against Virulent ILTV in SPF Chickens (FW311)

The efficacy of recombinant HVT/IBD-LTs FW311 was investigated in SPF chickens. Chickens one day of age were divided into three groups and chicks in Group 3 were vaccinated subcutaneously with approximately 3000 plaque forming units (pfu)/0.2 ml of of the recombinant HVT/IBD-LT FW311. Similarly, chicks in Group 2 were vaccinated subcutaneously with vaccine control (FW181). Chicks in Group 1 (non-immunized, challenged positive control) were left unvaccinated. The chickens were bled each week between 1 and 4 weeks of age. Challenge was conducted at 4 weeks of age. For challenge, 1×10³ EID₅₀ of virulent ILTV US strain was administered via intra-tracheal route. Chickens were observed daily for clinical signs associated with ILTV, such as rales, gasping, stretched neck, bloody expectorant, nasal exudate, foamy eyes, watery eyes, mucoid conjunctivitis, hemorrhagic nares, swollen head/eyes/face, head flicking, ruffled feathers, depression and death. Ten days post challenge, chickens were necropsied.

The results of efficacy are summarized in Table IV and Table V below.

TABLE IV
Protection of recombinant HVT/IBD-LT against virulent
ILTV challenge at 4 weeks of age in SPF chickens

| | | | Number of | Number of | |
|---|---|---|---|---|---|
| | | | chickens | chickens | |
| | | Number of | dead after | with | % |
| Group | Group | chickens | challenge | symptom | protection |
| 1 | NIC | 21 | 7 | 10 | 19 |
| 2 | FW181 | 23 | 0 | 0 | 100 |
| | (vaccine | | | | |
| | control) | | | | |
| 3 | FW311 | 26 | 0 | 2 | 92 |

The results show that FW311 elicits an excellent protective immunity against ILTV challenge infection in vivo.

TABLE V
Clinical sign scores of recombinant HVT/IBD-LT day by
day from virulent ILTV challenge (Efficacy trial)

| Group # | Group | 0 d | 1 d | 2 d | 3 d | 4 d | 5 d | 6 d | 7 d | 8 d | 9 d | 10 d |
|---|---|---|---|---|---|---|---|---|---|---|---|---|
| 1 | NIC | 0 | 0 | 0 | 1.05 | 2.65 | 2.94 | 2.76 | 1.53 | 0.13 | 0.43 | 0 |
| 2 | FW181 | 0 | 0 | 0 | 0 | 0 | 0 | 0 | 0 | 0 | 0 | 0 |
| | (vaccine | | | | | | | | | | | |
| | control) | | | | | | | | | | | |
| 3 | FW311 | 0 | 0 | 0 | 0 | 0.15 | 0.04 | 0 | 0 | 0 | 0 | 0 |

Clinical sign scores = total points/live birds

A very good clinical score is obtained in the group of chicken vaccinated with FW311 from day 3 after vaccination, with an average clinical sign score of 0.02.

Taken together, all these results show that the presence of an insulator in a multivalent rHVT increases the expression of the antigen placed under the influence of the insulator without altering the synthesis and expression of the second antigen. Moreover, the introduction of an insulator in the genome of a rHVT has no impact on the stability and the efficacy of the virus.

### Example 12: Efficacy of Recombinant HVT/IBD-LTs Against Virulent IBDV in SPF Chickens (FW313)

As for FW311 (example 8), the efficacy of recombinant HVT/IBD-LTs FW313 was investigated in SPF chickens. Chickens at one day of age were divided into three groups and chicks of Group 3 were vaccinated subcutaneously with approximately 3000 plaque forming units (pfu)/0.2 ml of the recombinant HVT/IBD-LT of the invention (FW313: Group 3,). Chicks in Group 2 (non-immunized, challenged positive control) were left unvaccinated. Chicks in Group 1 (non-immunized, non-challenged control) were left unvaccinated and unchallenged. The chickens were bled each week between 1 and 4 weeks of age. Challenge was conducted at 4 weeks of age. For challenge, 1×10³ EID₅₀ of virulent IBDV STC strain was administered via oral route. Chickens were observed daily for clinical signs associated with IBD, such as depression and death. Seven days post challenge, chickens were necropsied and observed for grossly observable bursal lesions such as edema, discoloration, atrophy, hemorrhage, and yellow or gelatinous exudates. Weights of body and bursa were also measured at necropsy for calculation of B/B index, which is the ratio between the weight of the bursa and the body weight of challenged birds divided by the same ratio of non-challenged birds.

The results of Efficacy trial are summarized Table IV below.

Construct FW313 induced the synthesis of anti-VP2 antibodies.

TABLE IV
Protection of recombinant HVT/IBD-LT against virulent IBDV challenge
at 4 weeks of age in SPF chickens (Efficacy trial 2)

| | | | | | Number of | |
|---|---|---|---|---|---|---|
| | | | | | chickens | |
| | | | | Number of | with bursal | |
| | | Number | B/B | chickens | lesions/total | % |
| | | of | Index | dead after | Number of | protection |
| Group | Group | chickens | (*) | challenge | chickens | (**) |
| 1 | NINC | 15 | 1.00 | 0 | 0 | 100 |
| 2 | NIC | 15 | 0.56 | 10 | 5 | 0 |
| 3 | FW313 | 15 | 0.92 | 0 | 2 | 87 |

(*) B/B index means Bursal-Body index
(**) % protection = 100-100 × (# dead + # lesion)/n.

The results confirm that FW313 is stable in vivo. The results further show that FW313 elicits a protective immunity against IBDV challenge infection in vivo.

1. A recombinant Marek's disease virus (rMDV) comprising, inserted into an insertion site, a recombinant nucleotide sequence encoding an antigen operably linked to a promoter and an insulator, wherein the insulator is positioned upstream of the promoter.

2. The rMDV of claim 1, wherein the insulator comprises one or more CCCTC-binding factor (CTCF) motif(s).

3. The rMDV of claim 1, wherein the insulator comprises a functional fragment of a murine 3 ′hypersensitivity site 1 (m3′HSl) insulator.

4. The rMDV of claim 3, wherein the functional fragment of m3′HSl comprises a nucleotide sequence as set forth in SEQ ID NO: 4, or a nucleotide sequence having at least 90% identity to the full length sequence as set forth in SEQ ID NO:4, and retaining an insulator activity.

5. The rMDV of claim 1, comprising a first recombinant nucleotide sequence encoding a first antigen inserted in a first insertion site, and a second recombinant nucleotide sequence encoding a second antigen inserted into a second insertion site, different from the first insertion site, wherein the first recombinant nucleotide sequence encoding an antigen is operably linked to a promoter and to an insulator positioned upstream of the promoter, and wherein the second recombinant nucleotide sequence is operably linked to a promoter, and wherein the nucleotide sequences encode different antigens.

6. (canceled)

7. The rMDV of claim 1, wherein the insertion site(s) is/are located in non-coding regions of the viral genome.

8. The rMDV of claim 1, wherein the recombinant nucleotide sequence(s) encodes an antigen from an avian pathogen, wherein the antigen(s) is/are chosen among an antigen of avian paramvxovirus type 1, an antigen of Gumboro disease virus, an antigen of the infectious laryngotracheitis virus (ILTV), an antigen of Mycoplasma gallisepticum, and an antigen of the avian influenza virus.

9. (canceled)

10. The rMDV of claim 1, wherein the promoter controlling the expression of a recombinant nucleotide sequence is chosen among the chicken beta-actin (Bac) promoter, the Pec promoter, the Murine Cytomegalovirus (Mcmv) immediate-early (ie) 1 promoter, the Human Cytomegalovirus promoter (Hcmv), the Simian virus (SV40) 40 promoter, and the Raus Sarcoma virus (RSV) promoter, or any fragments thereof which retain a promoter activity.

11. The rMDV of claim 1, wherein the rMDV is a recombinant herpes virus of turkey (rHVT).

12. The rMDV of claim 7, comprising a first recombinant nucleotide sequence inserted in the non-coding region located between UL44 and UL45, and a second recombinant nucleotide sequence inserted in the non-coding region located between UL45 and UL46, wherein the first recombinant nucleotide sequence encoding an antigen is operably linked to a promoter and to an insulator positioned upstream of the promoter, and wherein the second recombinant nucleotide sequence is operably linked to a promoter.

13. The rMDV claim 7, comprising a first recombinant nucleotide sequence inserted in the non-coding region located between UL44 and UL45, and a second recombinant nucleotide sequence inserted in the non-coding region located between UL45 and UL46, wherein the first recombinant nucleotide sequence encoding an antigen is operably linked to a promoter and wherein the second recombinant nucleotide sequence is operably linked to a promoter and to an insulator positioned upstream of the promoter.

14. The rMDV of claim 12, wherein the recombinant nucleotide sequence encodes a VP2 protein of IBDV or an antigenic fragment thereof, and the second recombinant nucleotide sequence encodes a gB protein of ILTV or an antigenic fragment thereof, on the reverse.

15. The rMDV of claim 11, wherein a first recombinant nucleotide sequence encoding a VP2 protein of IBDV or an antigenic fragment thereof operably linked to a Bac promoter and a m3′HSl insulator, or a functional fragment thereof, positioned upstream of the Bac promoter is inserted in a first insertion site located in the non-coding region between UL45 and UL46, and a second recombinant nucleotide sequence encoding a gB protein of ILTV or an antigenic fragment thereof operably linked to a promoter, is inserted in a second insertion site located in the non-coding region between UL44 and UL45, wherein the functional fragment of m3 ′HS 1 insulator comprises a nucleotide sequence having a CTCF motif and at least 90% identity to the full length sequence as set forth in SEQ ID NO:4, and retaining an insulator activity.

16. The rMDV of claim 10, wherein a first recombinant nucleotide sequence encoding a VP2 protein of IBDV or an antigenic fragment thereof operably linked to a Mcmv ie1 promoter, and a m3′HSl insulator, or a functional fragment thereof, positioned upstream of the Mcmv ie1 promoter is inserted in a first insertion site located in the non-coding region between UL44 and UL45, and a second recombinant nucleotide sequence encoding a gB protein of ILTV or an antigenic fragment thereof operably linked to a promoter is inserted in a the second insertion site located in the non-coding region between UL45 and UL46, wherein the functional fragment of m3 ′HS 1 insulator comprises or consists of a nucleotide sequence having a CTCF motif and at least 90% identity to the full length sequence as set forth in SEQ ID NO:4, and retaining an insulator activity.

17. The rMDV of claim 1, comprising a single recombinant nucleotide sequence encoding an antigen, said single recombinant nucleotide sequence being operably linked to a promoter and to an insulator positioned upstream of the promoter, and wherein the single recombinant nucleotide sequence is inserted in an insertion site located in the non-coding region between UL45 and UL46.

18. (canceled)

19. The rMDV of claim 15, wherein a recombinant nucleotide sequence encoding a VP2 protein of IBDV or an antigenic fragment thereof operably linked to a Bac promoter, and a functional fragment of m3′HSl insulator positioned upstream of the Bac promoter is inserted in an insertion site located in the non-coding region between UL45 and UL46, and wherein the functional fragment of m3′HSl insulator comprises a nucleotide sequence having a CTCF motif and at least 90% identity to the full length sequence as set forth in SEQ ID NO:4, and retaining an insulator activity.

20. The rMDV of claim 15, wherein a recombinant nucleotide sequence encoding a F protein of NDV or an antigenic fragment thereof operably linked to a Bac promoter, and a functional fragment of m3 ′HS 1 insulator positioned upstream of the Bac promoter is inserted in an insertion site located in the non-coding region between UL45 and UL46, and wherein the functional fragment of m3′HSl insulator comprises a nucleotide sequence having a CTCF motif and at least 90% identity to the full length sequence as set forth in SEQ ID NO:4, and retaining an insulator activity.

21. A host cell comprising a rMDV of claim 1 or a nucleic acid molecule comprising the genome of a rMDV of claim 1.

22. A vaccine composition comprising a rMDV of claim 1 and pharmaceutical acceptable vehicle.

23.-28. (canceled)

29. A method of immunizing or vaccinating an avian such as a poultry against an avian pathogen, by administering to said avian an immunologically effective amount of a rMDV of claim 1.

30. A method for increasing the onset of immunity in an avian such as a poultry against an avian pathogen, by administering to said avian an immunologically effective amount of a rMDV of claim 1.

31. The method of claim 29, wherein the rMDV is administered orally, ocularly, by oculo-nasal administration using aerosol, intranasally, by cloacal administration, by mucosal administration, in ovo, or by injection.
